(19) United States
(12) Patent Application Publication
(10) Pub. No.: US 2023/0087388 A1
(43) Pub. Date: Mar. 23, 2023

(54) INHIBITORS OF HUMAN DEUBIQUITINASES FOR THE TREATMENT OF CORONAVIRAL INFECTIONS

(71) Applicant: Immunologik GmbH, Berlin (DE)

(72) Inventors: Christian Setz, Memmelsdorf (DE); Ulrich Schubert, Trockenborn-Wolfersdorf (DE)

(21) Appl. No.: 17/802,886

(22) PCT Filed: Feb. 26, 2021
(86) PCT No.: PCT/EP2021/000023

§ 371 Date: Aug. 26, 2022

(30) Foreign Application Priority Data
Feb. 28, 2020 (EP) 20000089.1
Apr. 14, 2020 (EP) 20000148.5

(51) Int. Cl. A61K 31/381; A61P 31/14

(52) U.S. Cl. CPC A61K 31/381; A61P 31/14

(57) ABSTRACT

The present application relates to small molecule inhibitors of human deubiquitinases for use in the prevention or treatment of a coronaviral infection. It relates in particular to small molecule inhibitors of USP7 and USP47. Pharmaceutical compositions, respective advantageous formulation techniques and a method of treatment are disclosed.

The present application relates to small molecule inhibitors of human deubiquitinases for use in the prevention or treatment of a coronaviral infection. It relates in particular to small molecule inhibitors of USP7 and USP47. Pharmaceutical compositions, respective advantageous formulation techniques and a method of treatment are disclosed.

### BACKGROUND OF THE INVENTION

As a result of ecological, climatic and demographic changes, so-called ‘emerging’ viruses are increasingly being transmitted from their natural animal hosts to humans. Due to accelerated globalization they bear the risk of triggering a pandemic. Emerging viruses may cause acute and often life-threatening diseases. Coronaviridae have become notorious for such transmissions. Examples are Severe acute respiratory syndrome coronavirus (SARS-CoV) and Middle East respiratory syndrome-related coronavirus (MERS-CoV), and most recently, the outbreak of Severe acute respiratory syndrome coronavirus 2 (SARS-CoV-2; COVID-19) in Wuhan, China. A total of over 104 million SARS-CoV-2 infections with more than 2 million casualties worldwide have been reported by the Johns Hopkins University Coronavirus Resource Center, as of Feb. 4, 2021. The incubation period of SARS-CoV-2 ranges between two days and two weeks, in some cases up to one month.

Typical symptoms of SARS-CoV-2 are fever, cough, and shortness of breath. However, the infection can also cause severe pulmonary injury, leading to rapid onset of progressive malfunction of the lungs, especially with regard to the ability to take up oxygen. This is usually associated with the malfunction of other organs. This acute lung injury (ALI) condition is associated with extensive lung inflammation and accumulation of fluid in the alveoli. It is characterized by diffuse pulmonary microvascular injury resulting in increased permeability and, thus, non-cardiogenic pulmonary edema. In consequence, this leads to pathologically low oxygen levels in the lungs. Other common symptoms associated with COVID-19 patients in ICU care are pulmonary embolism, thrombosis, venous thromboembolism and brain ischemia.

Coronaviruses are primarily spread through close contact, in particular through respiratory droplets from coughs and sneezes. In contrast to the SARS-CoV and MERS-CoV, SARS-CoV-2 can be transmitted from human to human during the incubation period while the infected patient does not show yet any symptoms of disease. Moreover, SARS-CoV-2 can already replicate in the throat. In contrast, the receptors for SARS-CoV and MERS-CoV are located deep in the lungs. Thus, SARS-CoV-2 can be transmitted much easier from human to human in comparison to SARS-CoV and MERS-CoV which strongly increases the infection rate.

In general, coronaviruses (family Coronaviridae, group Coronaviruses) form a relatively diverse group of large, enveloped, positive strand RNA viruses, which can cause different types of diarrhea and respiratory diseases in humans and animals. They have a very narrow host range and replicate very poorly in cell culture. However, cell culture systems for SARS-CoV-2 could be successfully established.

Sequencing of SARS-CoV-2 revealed an approx. 29.8 kbp genome consisting of 14 open reading frame. Moreover, the virus is phylogenetically closely related to the SARS-CoV (89.1% nucleotide similarity) (cf. Wu et al. (2020) Nature 579: 265-269). Like other coronaviruses, SARS-CoV-2 enters the cell by endocytosis and membrane fusion. The viruses are released from the cell by the secretory pathway. The natural reservoir of the virus is unknown.

To date, no specific therapeutic options for the treatment of SARS-CoV-2 infections, respectively COVID-19 are established. Some success could be achieved with the antiviral drugs remdesivir, avifavir and favipiravir as well as with the anti-parasitic drug ivermectin. A nasal spray containing nanoantibodies against the SARS-CoV-2 spike protein is a promising development (AeroNabs). In severe stage COVID-19 patients the administration of the glucocorticoid dexamethasone showed to be effective.

A major problem is the high mutation rate of coronaviruses. So when for an effective pharmacological treatment for infections with a particular coronavirus a viral target is inhibited odds are high that this coronavirus soon becomes partially or completely resistant against this treatment. The same problem will occur as soon as the next coronavirus passes the species barrier to humans and another epidemic develops.

Thus, there is a strong medical need for an effective pharmacological treatment for patients infected with SARS-CoV-2 or similar coronaviruses and for limiting the current pandemic spread of this virus. Ideally, such a pharmacological treatment should also address the high mutation rate and offer at least a treatment option for future coronavirus outbreaks.

### DESCRIPTION OF THE INVENTION

Surprisingly, this task is solved by the small molecule human deubiquitinase inhibitors according to the disclosure. In particular small molecule inhibitors of the human deubiquitinases USP7 and/or USP47 show a good efficacy in the treatment of a coronaviral infection.

USP7 and USP47 are phylogenetically closely related. USP7 is mostly associated with tumors, as its inhibition reactivates the tumor suppressor p53 in many cancers. USP7 is also associated with the immune system (cf. Antao et al. (2020) Cancers 12: 1579). As far as being investigated until now compounds that show an inhibitory activity against USP7 inhibit in general also USP47. Therefore, it seems that USP47 acts like a tissue-specific isozyme of USP7.

Thus, the present disclosure refers to an inhibitor of human deubiquitinases USP7 and/or USP47 as well as its pharmaceutically acceptable salts, hydrates and solvates for use in the prophylaxis or treatment of a coronaviral infection.

Preferred embodiments of such USP7 and/or USP47 inhibitors are the pyridine-3,5-(bis)thiocyanates according to the disclosure.

Suitable pyridine-3,5-bis(thiocyanates) according to the disclosure are pyridine-3,5-(bis)thiocyanates according to the general formula (I)

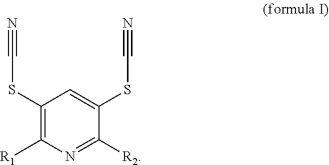

wherein

R₁ and R₂ each independently from one another is —H, —OH, —NHR₃, —NR₃R₄, a substituted or non-substituted linear or ramified alkyl residue with 1 to 3 carbon atoms, —CO—OCH₃, —CO—OC₂H₅, —CO—NH₂, —NH₂, —NO₂, —Cl, —Br, —F, or —SO₂H; and

R₃ and R₄ each independently from one another is —OH, —CH₃, —C₂H₅, —CH₂OH, —CHO, —COOH, —CO—CH₃, or —CO—NH₂.

In particular, 2,6-diaminopyridine-3,5-bis(thiocyanate) showed to be effective. This compound is mostly known as PR-619 (formula II) and is mostly preferred.

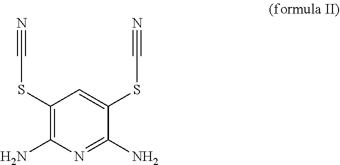 [SMILES: N#CSc1cc(SC#N)c(N)nc1N]

As it was shown in Example 1, 10 μM PR-619 is able to broadly inhibit the replication of SARS-CoV-2 in infected Vero-B4 cells. This finding is corroborated in the qRT-PCR experiments of Example 2. At this concentration PR-619 does not show cytotoxic effects, as could be seen in Example 3. Moreover, in an enzyme test PR-619 exerts a direct inhibitory action at the papain-like protease (PLpro) of SARS-CoV-2 which is essential for its replication (Example 4).

Other preferred embodiments of such USP7 and/or USP47 inhibitors are the disubstituted 4-nitro-thiophenes according to the disclosure.

Suitable disubstituted 4-nitro-thiophenes according to the disclosure are disubstituted 4-nitro-thiophenes according to the general formula (III)

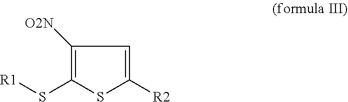

wherein

R₁ is phenyl, 2-chlorophenyl, 3-chlorophenyl, 4-chlorophenyl, 2,3-dichlorophenyl, 3,5-dichlorophenyl, 2,4-dichlorophenyl or 2,4-difluorophenyl, and

R₂ is acetyl, 1-hydroxyethyl, ethyl or n-butyl.

Compounds with these substituents of 4-nitro-thiophene were found to be effective in the inhibition of the human deubiquitinase USP7 (Chauhan et al. (2012) Cancer Cell 22: 345-358).

In particular, 1-[5-[(2,3-dichlorophenyl)thio]-4-nitro-2-thienyl]-ethanone showed to be effective. An alternative name is 1-[5-(2,3-dichlorophenyl)sulfanyl-4-nitro-2-thienyl]ethanone. This compound is mostly known as P005091, respectively P5091 (see formula IV) and is mostly preferred.

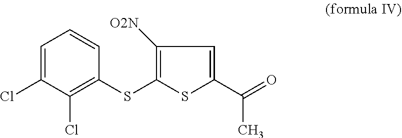 [SMILES: CC(=O)c1cc([N+](=O)[O-])c(Sc2cccc(Cl)c2Cl)s1]

In Example 5 it is described that 5 μM P5091 can nearly completely inhibit the replication of SARS-CoV-2 in infected Vero-B4 cells. At this concentration P5091 does not show cytotoxic effects, as shown in Example 6. Moreover, in an enzyme test P5091 exerts a direct inhibitory action at the papain-like protease (PLpro) of SARS-CoV-2 (Example 7).

Further suitable deubiquitinase inhibitors of USP7 and/or USP47 include P22077, ADC-01, ADC-03, HBX41108, HBX19818, HBX 28258, NSC 632839, NSC 144303, GNE-6640, GNE-6776, FT671, FT827, XL188, XL177a, XL024, XL058, XL041, 4-cyano-5-[(3,5-dichloro-4-pyridinyl)thio]-N-[4-(methylsulfonyl)phenyl]-2-thiophenecarboxamide and parthenolide.

The present disclosure refers also to the aforementioned deubiquitinase inhibitors of USP7 and/or USP47 for use in the prophylaxis or treatment of a coronaviral infection.

The deubiquitinase inhibitor P22077 (1-{5-[(2,4-difluorophenyl)sulfanyl]-4-nitro-2-thienyl}ethenone) was discovered by the company Progenra (Malvern, USA) and is commercially available. The description of syntheses of the substance class of P22077 was disclosed in WO 2010/114881. P22077 is specific for USP7 and USP47. There is a much lesser affinity towards ATXN3, BAP1 and USP1. P22077 causes an increase in polyubiquitin chains (Altun et al. (2011) Chem Biol 18: 1401-1412). An activation of the autophagy pathway could be shown (Seiberlich et al. (2013) Cell Biochem Biophys 67: 149-160). Up to now the investigation of P22077 focused on the treatment of tumors, without proving a therapeutically useful anti-proliferative potential for P22077. Further, P22077 was highly effective to repress the replication of HIV-1 primary T cells and macrophages and ex vivo preparations (human lymphoid aggregate culture, HLAC). The MHC-I antigen presentation of HIV-1 structural proteins was increased (cf. WO 2016/004915).

Almac Discovery discovered a family of piperidine derivatives that are specific USP7 inhibitors (WO 2018/073602 A1). Among them, ADC-01 and ADC-03 are the most promising compounds (Gavory et al. (2015) Cancer Res 75: 15) These compounds shall be therapeutically developed for oncological and immuno-oncological use.

HBX41108 (7-chloro-9-oxo-9H-indeno[1,2-b]pyrazine-2,3-dicarbonitrile), HBX19818 (N-(3-(benzyl(methyl)amino)propyl)-9-chloro-5,6,7,8-tetrahydroacridine-2-carboxamide) and HBX28258 (9-chloro-N-[3-[ethyl(phenylmethyl)amino]propyl]-5,6,7,8-tetrahydro-2-acridinecarboxamide) inhibit specifically USP7. These related compounds shall be therapeutically developed for oncological and immuno-oncological use (Reverdy et al. (2012) Chem Biol 19: 467-477).

GNE-6640 (CS 2824; 4-[2-amino-4-ethyl-5-(1H-indazol-5-yl)-3-pyridinyl]-phenol) and GNE-6776 (CS2823; 6′-amino-4′-ethyl-5′-(4-hydroxyphenyl)-N-methyl-[3,3′-bipyridine]-6-carboxamide) are USP7 inhibitors. GNE-6640 inhibits also USP43 and Ub-MDM2 while GNE-6776 is highly specific for USP7. These compounds induce tumor cell death and cytotoxicity by attenuating ubiquitin binding and thus USP7 activity (Kategaya et al. (2017) Nature 550: 534-538).

FT671 (5-[[1-[(3S)-4,4-difluoro-3-(3-fluoro-1H-pyrazol-1-yl)-1-oxobutyl]-4-hydroxy-4-piperidinyl]methyl]-1-(4-fluorophenyl)-1,5-dihydro-4H-Pyrazolo[3,4-d]pyrimidin-4-one) and FT827 (N-[4′-[[4-[(1,4-dihydro-1-methyl-4-oxo-5H-pyrazolo[3,4-d]pyrimidin-5-yl)methyl]-4-hydroxy-1-piperidinyl]carbonyl][1,1-biphenyl]-2-yl]-ethenesulfonamide) inhibit USP7 with high affinity and specificity. By this mechanism they lead to the re-activation of the tumor suppressor p53 in various cancers (Turnbull et al. (2017) Nature 550: 481-486).

WO 2019/067503 A1 discloses a number of related USP7 inhibitors to interact with tumor suppressor p53 for tumor therapy: ((R)-7-Chloro-3-((4-hydroxy-1-(3-phenylbutanoyl)piperidin-4-yl)methyl)quinazolin-4(3H)-one, (7-Chloro-3-((1-(3-phenylpropanoyl)piperidin-4-yl)methyl)quinazolin-4(3H)-one, 4-((7-Chloro-4-oxoquinazolin-3(4H)-yl)methyl)-1-(3-phenylpropanoyl)piperidine-4-carbonitrile, (7-Chloro-3-((4-hydroxy-1-(3-phenylpropyl)piperidin-4-yl)methyl)quinazolin-4(3H)-one, (7-chloro-3-((3-hydroxy-1-(3-phenylpropanoyl)pyrrolidin-3-yl)methyl)quinazolin-4(3H)-one, (3-((1-Acetyl-4-hydroxypiperidin-4-yl)methyl)-7-chloroquinazolin-4(3H)-one, (7-Chloro-3-((4-hydroxy-1-(2-phenylacetyl)piperidin-4-yl)methyl)quinazolin-4(3H)-one, (7-Chloro-3-((4-hydroxy-1-(3-phenylbutanoyl)piperidin-4-yl)methyl)quinazolin-4(3H)-one, ((S)-7-Chloro-3-((4-hydroxy-1-(3-phenylbutanoyl)piperidin-4-yl)methyl)quinazolin-4(3H)-one, ((R)—N-(3-((4-Hydroxy-1-(3-phenylbutanoyl)piperidin-4-yl)methyl)-4-oxo-3,4-dihydroquinazolin-7-yl)-3-(4-methylpiperazin-1-yl)propenamide, ((S)—N-(3-((4-Hydroxy-1-(3-phenylbutanoyl)piperidin-4-yl)methyl)-4-oxo-3,4-dihydroquinazolin-7-yl)-3-(4-methylpiperazin-1-yl)propenamide, (N-(3-((4-Hydroxy-1-(3-phenylbutanoyl)piperidin-4-yl)methyl)-4-oxo-3,4- dihydroquinazolin-7-yl)-3-(4-methylpiperazin-1-yl)propenamide, (N-(3-((4-Hydroxy-1-(4-methyl phenylpentanoyl)piperidin-4-yl)methyl)-4-oxo-3,4-dihydroquinazolin-7-yl)-3-(4-methylpiperazin-1-yl)propenamide, ((R)—N-(3-((4-hydroxy-1-(3-phenylbutanoyl)piperidin yl)methyl)-4-oxo-3,4-dihydroquinazolin-7-yl)-3-morpholinopropanamide, ((R)-3-(Dimethylamino)-N-(3-((4-hydroxy-1-(3-phenylbutanoyl)piperidin-4-yl)methyl)-4-oxo-3,4-dihydroquinazolin-7-yl)propenamide, (N-(3-((4-hydroxy-1-(3-phenylbutanoyl)piperidin-4-yl)methyl)-4-oxo-3,4-dihydroquinazolin-7-yl)-3-(1H-imidazol-1-yl)propenamide, ((TST-(3-((4-hydroxy-1-(3-phenylbutanoyl)piperidin-4-yl)methyl)-4-oxo-3,4-dihydroquinazolin-7-yl)-3-(piperidin-1-yl)propenamide.

((R)—N-(3-((4-Hydroxy-1-(3-phenylbutanoyl)piperidin-4-yl)methyl)-4-oxo-3,4-dihydroquinazolin-7-yl)-3-(4-methylpiperazin-1-yl)propenamide is also known as XL188 (see Lamberto et al. (2017) Cell Chem Biol 24: 1490-1500).

Derived from XL188 is XL177a ((S)—N-(4-benzyl-5-(4-hydroxy-44(7-(3-(4-methylpiperazin-1-yl)propanamido)-4-oxoquinazolin-3(4H)-yl)methyl)piperidin-1-yl)-5-oxopentyl)-9-chloro-5,6,7,8-tetrahydroacridine-3-carboxamide). To this group belong also XL112, XL024 (((S)—N-(4-benzyl-5-(4-((7-(3-(4-methylpiperazin-1-yl)propanamido)-4-oxoquinazolin-3(4H)-yl)methyl)piperidin-1-yl)-5-oxopentyl)-9-chloro-5,6,7,8-tetrahydroacridine-3-carboxamide), XL058 (((S)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-5,6,7,8-tetrahydroacridine-3-carboxamide) and XL041 (((S)—N-(5-(4-hydroxy-4-((7-(3-(4-methylpiperazin-1-yl)propanamido)-4-oxoquinazolin-3(4H)-yl)methyl)piperidin-1-yl)-5-oxopentyl)-9-chloro-5,6,7,8-tetrahydroacridine-3-carboxamide) (cf. Schauer et al. (2020) Nature Sci Rep 10: 5324).

NSC 632839 (synonym: F6; 4-piperidione,3,5-bis[(4-methylphenyl)methylene]-hydrochloride) and NSC 144303 (4H-thiopyran-4-one-tetrahydro-3,5-bis[(4-nitrophenyl)methylene]-,1,1-dioxide) were found to sustain caspase 3/caspase 7 activity in the absence of a functional caspase-9. This includes a Bcl-2-dependent but apoptosome-independent mitochondrial pathway. Apoptosis seems to be introduced by the ubiquitin-proteasome system. These two compounds inhibit USP2, USP7 and the SENP2 deSUMOylase that does not affect the proteasomal proteolytic activity. They are developed as an alternative for cancer treatment (cf. Aleo et al. (2006) Cancer Res 66: 9235-9244; Nicholson et al. (2008) Protein Sci 17: 1035-1043).

4-cyano-5-[(3,5-dichloro-4-pyridinyl)thio]-N-[4-(methylsulfonyl)phenyl] thiophenecarboxamide is a deubiquitinase inhibitor that is specific for USP7 and USP47 over USP2, USP5, USP8, USP21, USP28, caspase-3, and cathepsin B. It inhibits the growth of HCT116 cells (cf. Weinstock et al. (2012) ACS Med Chem Lett 3: 789-792).

Parthenolide (1aR,4E,7aS,10aS,10bR)-2,3,6,7,7a,8,10a,10b-octahydro-1a,5-dimethyl methylene-oxireno[9,10]cyclodeca[1,2-b]furan-9(1aH)-one) is a sesquiterpene lactone found in Tanacetum parthenium. Originally, parthenolide was classified as HDAC1 inhibitor and NF-κB modulator. Anti-inflammatory and anti-hyperalgesic properties and effects against Leishmaniosis amazonensis infections were described. Recently, parthenolide was found to inhibit specifically USP7, Wnt signaling and therewith colorectal cancer cell growth (Li et al. (2020) J Biol Chem 295: 3576-3589).

It is known that coronaviruses use the protease PLpro, respectively their functional analogue to interfere with the proteasome systems of the human host cells in order to functionalize the respective cellular enzymes for their replication.

Proteasomes are multi-catalytic enzyme complexes accounting for ca. 1% of total cell protein. It represents the main proteolytic component in the cellular nucleus and the cytosol of eukaryotic cells. Proteasomes play many important roles in the cellular metabolism. A major function is the proteolysis of misfolded non-functional proteins. Another important function is the degradation of cellular and viral proteins for the T cell-mediated immune response and thus the generation of peptide ligands to be loaded upon MHC-I molecules (MHC=major histocompatibility complex). A subform is the immunoproteasome that is constitutively expressed in specific cell types, for example in the spleen, lymph nodes and antigen-presenting cells.

Substrates of proteasomes are usually marked for degradation by the attachment of ubiquitin oligomers. Ubiquitin (Ub) is a highly conserved, 76 amino acid long protein that is covalently coupled to the respective target protein. Ubiquitinylation is a reversible process. Ub molecules can be removed from the target protein by numerous deubiquitinases (deubiquitinating enzymes, DUBs). Thus Ub molecules become again intracellularly available. This recycling process is essential for cell homeostasis. This regulatory system of ubiquitinylation of target proteins and proteasomal proteolysis is usually referred to as ubiquitin proteasome system (UPS).

DUBs are a broad class of Ub hydrolases and are the intracellular opponents of ubiquitin E3 ligases. The target proteins can be deubiquitinated completely or partially. In humans, more than 100 DUBs are known until now, which are subdivided into five families:
ubiquitin-specific protease family (USP)
ubiquitin C-terminal hydrolases (UCHs)
ovarian tumor proteases (OTUs)
Josephin family
JAB1/MPN/Mov34 family (JAMMs)

The first four are cysteine proteases while the last one is a zinc metalloprotease.

The most important cellular functions of DUBs are:

1. They are crucial for the new generation of free ubiquitin. Ubiquitin is a linear fusion protein encoded on several genes and consisting of a row of Ub monomers. After translation this ubiquitin chain is specifically hydrolyzed by DUBs so that a release of free Ub molecules is effected.

2. They remove in a highly specific manner polyubiquitin chains of post-translationally modified proteins. Thus the target protein is stabilized. Furthermore the DUBs POH1, UCH37 and USP14 that are associated with the proteasome remove the ubiquitin chains from proteins that have already entered the proteasome for proteolysis. This way the content of free ubiquitin is kept in balance in the cell.

3. They alter the ubiquitin modifications of proteins by trimming the existing ubiquitin chains. For example, an originally polyubiquitinated protein can bear only a single ubiquitin in the end. The function of the protein may be completely different thereby.

In most cases DUBs show a high specificity for certain substrates, tissues as well as for specific ubiquitin chains.

DUBs, however, undergo themselves a complex regulation. Thus post-translational modifications such as phosphorylation, ubiquitination or SUMOylation may occur, leading to the activation or deactivation of the respective DUBs.

Also DUBs can undergo a conformational change by binding to certain proteins. This can also lead to the activation or deactivation of the respective DUBs.

Certain DUBs are limited in their activity to certain cell compartments. If needed, they are transported there.

Thus, DUBs are an interesting target for influencing cellular regulatory process through their inhibition or through the modification of their activity. Also for clinical applications the whole UPS has moved into the focus in the last years. Thus, it was for example tried in the last years to inhibit components of the UPS such as the 26S proteasome or Ub ligases by means of small molecules. The rationale therefor was above all to find new therapeutic approaches for tumors. The only medications hitherto approved are the proteasome inhibitors Bortezomib (Velcade®) for the treatment of multiple myeloma and mantle cell lymphoma, carfilzomib (Kyprolis®) and ixazomib (Ninlaro®) for multiple myeloma only. The mechanism of action, however, is not specific. The global effectiveness concerns numerous vital process, causing partially serious side effects such as the manifestation of a peripheral neuropathy with pain and numbness particularly in the extremities. In the clinical development of E3 ligases inhibitors there is no breakthrough until now.

Also in the investigation of DUB inhibitors the development of new therapeutic approaches in tumor treatment is in the focus of interest. The DUB inhibitors known until now act in general highly specifically on a certain DUB target. Therefore, they display a relatively small cytotoxicity. Clearly less and milder side effects in patients are expected than in the use of 26S proteasome inhibitors or E1 ligase inhibitors

For some DUBs oncogenic properties have been shown, for example for USP2a, USP7, USP20 and USP33. Thus, the inhibition of DUBs as a mode of action is regarded as a method for blocking or at least reducing the oncogenic properties of these DUBs.

PR-619 (2,6-diaminopyridine-3,5-(bis)thiocyanate) inhibits USP1, USP2, USP4, USP5, USP7, USP8, USP9X, USP10, USP14, USP15, USP16, USP19, USP20, USP22, USP24, USP28, USP47, USP48, UCH-L1, UCH-L3, UCH-L5/UCH37, ATXN3, BAP1, JOSD2, OTUD5, VCIP135 and YOD. The application of PR-619 leads to an increase in polyubiquitin chains (Altun et al. (2011) Chem Biol 18: 1401-1412). Until now the research on PR-619 has focused on investigations of tumor treatment, without a therapeutically useful anti-proliferative potential to be found until now. PR-619 showed to be strikingly effective to repress the replication of HIV-1 in primary T cells and macrophages and ex vivo preparations (human lymphoid aggregate culture, HLAC) via an inhibition of USP47. The MHC-I antigen presentation of HIV-1 structural proteins was increased (cf. WO 2016/004917).

Thus, the present application relates also to a pyridine-3,5-(bis)thiocyanate according to the disclosure as well as their pharmaceutically acceptable salts, hydrates and solvates for use in the prophylaxis or treatment of a coronaviral infection.

In particular, the present application relates to PR-619 for use in the prophylaxis or treatment of a coronaviral infection.

In CB-17 mice the use of the DUB inhibitor P5091 led to apoptosis in multiple myeloma cells. A development of resistance under the proteasome inhibitor Bortezomib could thus be reverted. It was described that P5091 induces apoptosis in tumor cell lines (Chauhan et al. (2012) Cancer Cell 22: 345-358). P5091 inhibits potently and specifically the closely related deubiquitinases USP7 and USP 47 without blocking proteasome activity (Chauhan et al. (2012) Cancer Cell 22: 345-358; Weinstock et al. (2012) ACS Med Chem Lett 3: 789-792).

Thus, the present application relates also to a disubstituted 4-nitro-thiophene according to the disclosure as well as their pharmaceutically acceptable salts, hydrates and solvates for use in the prophylaxis or treatment of a coronaviral infection.

In particular, the present application relates to P5091 for use in the prophylaxis or treatment of a coronaviral infection.

Coronaviral infections that can be treated with the deubiquitinase inhibitors according to the disclosure are above all infections with the highly pathogenic SARS-CoV; MERS-CoV and SARS-CoV-2.

But also infections with less pathogenic coronaviridae as listed in the following can be thus treated. The terms “coronavirus” or “coronaviral” refer mainly to the sub-family of orthocoronavirinae. They are subdivided into the genera of alphacoronaviruses, betacoronaviruses, gammacoronaviruses and deltacoronaviruses. Alphacoronaviruses comprise the sub-genera of colacoviruses (species: bat coronavirus CDPHE15), decaviruses (bat coronavirus HKU10, Rhinolophus ferrumequinum alphacoronavirus HuB-2013), duvinacoviruses (human coronavirus 229E), luchacoviruses (Lucheng Rn rat coronavirus), minacoviruses (Ferret coronavirus, Mink coronavirus 1), minunacoviruses (miniopterus bat coronavirus 1, miniopterus bat coronavirus HKU8), myotacoviruses (Myotis ricketti alphacoronavirus Sax-2011), nylactoviruses (Nyctalus velutinus alphacoronavirus SC-2013), pedacoviruses (porcine epidemic diarrhea virus, Scotophilus bat coronavirus 512), rhinacoviruses (Rhinolophus bat coronavirus HKU2), setracoviruses (human coronavirus NL63, NL63-related bat coronavirus strain BtKYNM63-9b) and tegacoviruses (Alphacoronavirus 1-type species). Betacoronaviruses comprise the sub-genera of embecoviruses (Betacoronavirus 1 (subspecies: human coronavirus 0C43), China Rattus coronavirus HKU24, human coronavirus HKU1, murine coronavirus-type species), hibecoviruses (Bat Hp-betacoronavirus Zhejiang 2013), merbecoviruses (Hedgehog coronavirus 1, MERS-CoV), Pipistrellus bat coronavirus HKU5, Tylonycteris bat coronavirus HKU4), nobecoviruses (Rousettus bat coronavirus GCCDC1, Rousettus bat coronavirus HKU9 and sarbecoviruses (severe acute respiratory syndrome-related coronavirus (subspecies: SARS-CoV, SARS-CoV-2). Gammacoronaviruses comprise the sub-genera of cegacoviruses (Beluga whale coronavirus SW1) and igacoviruses (Avian coronavirus-type species). Deltacoronaviruses comprise the sub-genera of andecoviruses (Wigeon coronavirus HKU20), buldecoviruses (Bulbul coronavirus HKU11-type species, Porcine coronavirus HKU15, Munia coronavirus HKU13, White-eye coronavirus HKU16), herdecoviruses (Night heron coronavirus HKU19) and Moordecoviruses (Common moorhen coronavirus HKU21).

Coronaviruses pathogenic in humans are until now SARS-CoV, SARS-CoV-2, MERS-CoV and HCoV-HKU1, HCoV-NL-63, HCoV-0C43 and HCoV-229E. The last four cause only relatively mild symptoms (cf. Andersen et al.: The Proximal Origin of SARS-CoV-2, on virologica.org, as of Feb. 17, 2020).

Thus, the present application relates in particular to the human deubiquitinase inhibitors of USP7 and/or USP47 according to the disclosure or one of their pharmaceutically acceptable salts for use in the prophylaxis or treatment of a coronaviral infection, wherein said coronaviral infection is selected from a group comprising a SARS-CoV, SARS-CoV-2, MERS-CoV, HCoV-HKU1, HCoV-NL-63, HCoV-0C43 and HCoV-229E infection.

More preferred is a pyridine-3,5-(bis)thiocyanate according to the disclosure or one of their pharmaceutically acceptable salts for use in the prophylaxis or treatment of a coronaviral infection, wherein said coronaviral infection is SARS-CoV-2.

Still more preferred is PR-619 or one of its pharmaceutically acceptable salts for use in the prophylaxis or treatment of a coronaviral infection, wherein said coronaviral infection is SARS-CoV-2.

More preferred is a disubstituted 4-nitro-thiophene according to the disclosure or one of their pharmaceutically acceptable salts for use in the prophylaxis or treatment of a coronaviral infection, wherein said coronaviral infection is SARS-CoV-2.

Still more preferred is P5091 or one of its pharmaceutically acceptable salts for use in the prophylaxis or treatment of a coronaviral infection, wherein said coronaviral infection is SARS-CoV-2.

The other above-mentioned animal coronaviruses have not yet made a transfer to humans (zoonosis), but this may happen in the future with an unpredictable pathology. Thus, the scope of the present application relates also to a small molecule inhibitor of human deubiquitinases according to the disclosure for use in the prophylaxis or treatment of these animal coronaviral infections in animals and humans.

The concept for treating coronaviral infections over all species is based on the structural similarity of coronaviruses. Thus, it can be assumed that treatment and/or prevention options can be transferred from one coronavirus to another. Coronavirus particles contain four main structural proteins: spike (S), membrane (M), envelope (E) and nucleocapsid (N), all of which are encoded within the 3′ end of the viral genome.

Coronaviruses contain a non-segmented, positive-sense RNA genome of ˜30 kb. The genome contains a 5′ cap structure along with a 3′ poly (A) tail, allowing it to act as an mRNA for translation of the replicase polyproteins. The replicase gene encoding the nonstructural proteins (nsps) occupies two-thirds of the genome, about 20 kb, as opposed to the structural and accessory proteins, which make up only about 10 kb of the viral genome. The organization of the coronavirus genome is 5′-leader-UTR-replicase-S(Spike)-E (Envelope)-M (Membrane)-N (Nucleocapsid)-3′ UTR-poly (A) tail with accessory genes interspersed within the structural genes at the 3′ end of the genome. The accessory proteins are almost exclusively nonessential for replication in tissue culture; however, some have been shown to have important roles in viral pathogenesis (cf. Zhao et al. (2012) Cell Host Microbe 11: 607-616).

The coronavirus life cycle starts with an initial attachment of the virion to the host cell by interactions between the S protein and its receptor. The sites of receptor binding domains (RBD) within the S1 region of a coronavirus S protein vary depending on the virus. The S-protein-receptor interaction is the primary determinant for a coronavirus to infect a host species and also governs the tissue tropism of the virus. Many coronaviruses utilize peptidases as their cellular receptor. It is unclear why peptidases are used, as entry occurs even in the absence of the enzymatic domain of these proteins. Many alphacoronaviruses utilize aminopeptidase N (APN) as their receptor, many betacoronaviruses such as SARS-CoV, SARS-CoV-2 and HCoV-NL63 use angiotensin-converting enzyme II (ACE2) receptors, MHV enters through CEACAM1, and MERS-CoV binds to dipeptidyl-peptidase 4 (DPP4) to gain entry into human cells. Following receptor binding, the virus must next gain access to the host cell cytosol. This is generally accomplished by acid-dependent proteolytic cleavage of S protein by a cathepsin, TMPRRS2 or another protease, followed by fusion of the viral and cellular membranes, and ultimately the release of the viral genome into the cytoplasm.

Coronaviruses encode either two or three proteases that cleave the replicase polyproteins. They are the papain-like proteases (PLpro), encoded within nsp3, and a serine type protease, the main protease, or Mpro, encoded by nsp5. Most coronaviruses encode two PLpros within nsp3, except the gam macoronaviruses, SARS-CoV and MERS-CoV, which only express one PLpro (Mielech et al. (2014) Virus Res doi: 10.1016).

This papain-like protease (PLpro) was found in SARS-CoV to act the same way as a deubiquitinase within the human cellular ubiquitin proteasome system (UPS) (cf. Raaben et al. (2010) J Virol 84: 7869-7879). PLpro in SARS-CoV-2 has a very high homology with SARS-CoV (96.1%, Nguyen et al. (2020) https://doi:org/10:1101/2020.02.05.936013). These PLpro are a target for DUB inhibitors. For example, GRL0617 (5-amino-2-methyl-N-[(1R)-1-(1-naphthalenyl)ethyl]benzamide) was found to inhibit PLpro in SARS-CoV. Thus, GRL0617 can be regarded as a DUB inhibitor against coronaviral targets (cf. Ratia et al. (2008) PNAS 105: 16119-16124).

Chloroquine ((RS)—N′-(7-chloroquinolin-4-yl)-N,N-diethyl-pentane-1,4-diamine) is a long-known malaria medication acting as an autophagy inhibitor. Chloroquine acts synergistically with bortezomib to suppress cell proliferation and induce apoptosis in tumor models (Hui et al. (2012) Cancer 118: 5560-5571). Chloroquine is known to block viral infections by increasing endosomal pH requires for virus/cell fusion and it interferes with the glycosylation of ACE II receptors in SARS-CoV infections (Vincent et al. (2005) Virol J 2, 69). Recently, it was found that a combination of the antiviral drug remdesivir and chloroquine inhibit SARS-CoV-2 in vitro (Wang et al. (2020) Cell Res 0: 1-3).

Proteins designated for degradation by the proteasome are marked by a successive linking of ubiquitin molecules to these proteins. This is effected by a cascade of ligases E1-E3. The longer the ubiquitin chain the better is the respective protein recognized at the proteasome, respectively the marked protein is longer available for degradation by the proteasome.

The counterpart of the ligases are the deubiquitinases. They cleave ubiquitin molecules from this ubiquitin chain in a substrate- and tissue-specific manner. Ligases and deubiquitinases are in a constant balance and thus regulate protein degradation in the cell.

If a protein marked with a ubiquitin chain is recognized by the 26S proteasome it docks at the ubiquitin recognition site at the entrance of the proteasome and is proteolytically degraded into amino acids in consecutive stages. At the end, the amino acids and the ubiquitin molecules leave the proteasome and are available for recycling by the cell.

In a general picture, the proteasome can be compared to a collaboratively organized factory while ligases and deubiquitinases like PR-619 are part of the supply chain. The proteasome is a functional unit of many proteins and accordingly quite large. In contrast, more than a hundred of different cellular deubiquitinases have been described that are independent proteins and not a part of the proteasome.

A proteasome inhibitor inhibits the whole proteasome while ligases or deubiquitinases not, et vice versa. Taken together, a proteasome inhibitor suppresses the cellular degradation of proteins.

In contrast, a deubiquitinase inhibitor prevents in a substrate- and tissue-specific manner that a particular deubiquitinase cleaves ubiquitin molecules from a thus marked protein. In consequence, the ubiquitin chain is relatively prolonged. Therefore, the respective protein is longer available for the proteasome and is better recognized. Deubiquitinase inhibitors act much more specifically and therefore show much less cytotoxicity in the effective dosage range. Taken together, a deubiquitinase inhibitor promotes the cellular degradation of proteins.

Thus, proteasome inhibitors and deubiquitinase inhibitors act mechanistically in opposite directions.

For an effective treatment of coronaviral infections it may be advantageous to provide to a patient in need thereof a combinational therapy by combining an inhibitor of human deubiquitinases according to the disclosure with at least one antiviral agent.

For example, from HIV, respectively anti-retroviral therapy the following classes are known:

Reverse transcriptase inhibitors suitable for such a combination therapy are nucleoside reverse transcriptase inhibitors (NRTI) and non-nucleoside reverse transcriptase inhibitors (NNRTI). Examples of NRTI include, but are not limited to, abacavir, didanosine, emtricitabine, lamivudine, stavudine, tenofovir, zidovudine, zalcitabine, entecavir, adefovir, elvucitabine, fosalvudine(-tidoxil), fozivudintidoxil, lagiciclovir, alamifovir, clevudine, pradefovir, telbivudine. Examples of NNRTI include, but are not limited to, efavirenz, etravirine, nevirapine, rilpivirine, delavirdine, emivirine, lersivirine.

Suitable for a combination therapy according to the invention are integrase inhibitors such as raltegravir, elvitegravir, dolutegravir, MK-2048.

Examples of HIV protease inhibitors suitable for a combination therapy according to the invention are saquinavir, indinavir, ritonavir, nelfinavir, amprenavir, lopinavir, atazanavir, fosamprenavir, tipranavir, darunavir, brecanavir, mozenavir, tipranavir.

Examples of entry inhibitors suitable for a combination therapy according to the invention are enfuvirtide and maraviroc.

Further, general virostatic agents suitable for a combination therapy according to the invention can be selected from the group comprising ancriviroc, aplaviroc, cenicriviroc, enfuvirtide, maraviroc, vicriviroc, amantadine, rimantadine, pleconaril, idoxuridine, aciclovir, brivudine, famciclovir, penciclovir, sorivudine, valaciclovir, cidofovir, ganciclovir, valganciclovir, sofosbusvir, foscarnet, ribavirine, taribavirine, filibuvir, nesbuvir, tegobuvir, fosdevirine, favipiravir, merimepodib, asunaprevir, balapiravir, boceprivir, ciluprevir, danoprevir, daclatasvir, narlaprevir, telaprevir, simeprevir, vanipevir, rupintrivir, remdesivir, fomivirsen, amenamevir, alisporivir, bevirimat, letermovir, laninamavir, oseltamivir, peramivir, zanamivir.

General immunostimulatory agents suitable for a combination therapy according to the invention can be selected from the group comprising interferons (alpha-, beta-, gamma-, tau-interferon), interleukins, CSF, PDGF, EGF, IGF, THF, levamisol, dimepranol, inosine.

Furthermore, possible combinations according to the invention include adjuvants such as cobicistat.

The terms “medicine” or “medical” comprise human as well as veterinary medicine.

The term “organism” refers to a living being, especially a human or an animal, possessing a self-regulating immunological system.

The term “host organism” is used in terms of the application for those organisms exploited for replication by viruses, herein especially retroviruses, following an infection with them.

The term “active agent” in this application refers to a deubiquitinase inhibitor according to the disclosure for use according to the disclosure. Moreover, this term can comprise further pharmaceutical agents, known from the state of the art.

The terms “composition” and “pharmaceutical composition” comprise at least one deubiquitinase inhibitor according to the disclosure in any pharmacologically suitable defined dose and dosage form together with at least one suitable excipient or carrier substance as well as all substances which are directly or indirectly generated as a combination, accumulation, complex formation or crystal of the aforementioned ingredients, or come into being as a result of other reactions or interactions as well as optionally at least one further pharmaceutical agent known in the state of the art.

The term “excipient” is used in this application to describe each component of a pharmaceutical composition in addition to the active agent. The selection of a suitable excipient depends on factors such as dosage form and dose as well as the influence on the solubility and stability of the composition by the excipient itself.

The term “action” describes the inherent specific mode of action of the respective agent in the scope of the present application.

The terms “effect”, “therapeutic effect”, “action”, “therapeutic action” regarding at least one active agent according to the invention refer to causally occurring beneficial consequences for the organism, to which the at least one active agent has been administered.

In terms of the application, “therapeutically effective dose” means that a sufficient dose of the at least one deubiquitinase inhibitor according to the disclosure is administered to a living being or to a patient in need of such a treatment.

The terms “joint administration”, “combined administration” or “simultaneous administration” of at least one pharmaceutical agent according to the disclosure and/or of at least one pharmaceutical agent from the state-of-the-art comprise the administration of the mentioned agents at the same time or at time points factually related close to each other, as well as administrations of said agents at different times within a coherent experiment. The chronological order of the administration of said agents is not limited by these terms. Those skilled in the art will have no difficulties to deduce the described administrations in respect to their chronological or local order from his knowledge and experience.

The term “living being” refers to every animal, especially vertebrate, including human. A “patient” in terms of the application is a living being who suffers from a definable and diagnosable disease, and to whom a suitable active agent can be administered.

The terms “prophylaxis”, “treatment” and “therapy” comprise the administration of at least one deubiquitinase inhibitor according to the disclosure, alone or in combination with at least one further pharmaceutical agent known in the art, to a living being, in order to prevent the development of a certain disease, to inhibit, and to alleviate the symptoms, or to initiate a healing process of the respective disease.

The compounds according to the disclosure can be provided as pharmaceutically acceptable salts of organic and inorganic acids. Suitable examples are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, digluconic acid, lactic acid, tartaric acid, hydroxymaleic acid, pyruvic acid, phenylacetic acid, benzoic acid, p-aminobenzoic acid, p-hydroxybenzoic acid, dinitrobenzoic acid, chlorbenzoic acid, methanesulfonic acid, ethanesulfonic acid, nitric acid, hydroxyethanesulfonic acid, ethylenesulfonic acid, p-toluylsulfonic acid, naphthylsulfonic acid, sulfanilic acid, camphorsulfonic acid, alginic acid, capric acid, hippuric acid, pectinic acid, phthalic acid, quinic acid, mandelic acid, o-methyl mandelic acid, hydrogen benzenesulfonic acid, picric acid, adipic acid, cyclopentane propionic acid, D-o-toluyl tartaric acid, tartronic acid, benzenesulfonic acid, alpha-methyl benzoic acid, (o, m, p-)methyl benzoic acid, naphthylamine sulfonic acid, as well as salts from other mineral acids or carbonic acids well known to a person skilled in the art. These salts are generated by contacting the free base with a sufficient amount of the respective acid in order to build the salt in a conventional manner.

Pharmaceutically acceptable salts should be seen in terms of this application as an active agent containing a compound according to the invention in form of a salt, in particular if this salt bestows specific or ameliorated pharmacokinetic properties in comparison to the free form of the active agent or to another salt of the active agent. The pharmaceutically acceptable salt of the active agent may also bestow a pharmacokinetic characteristic to the active agent it did not have in its free form. Thus, it may even positively influence the pharmacodynamics of the active agent in respect to its therapeutic efficacy in the organism.

The compounds according to the disclosure can also be provided as hydrates or solvates. In terms of this application solvates refer to such forms of the compounds according to the invention that build a complex through coordination with solvent molecules. Hydrates are a specific form of solvates in which the coordination is effected by water molecules.

The at least one deubiquitinase inhibitor according to the disclosure or a drug combination according to the disclosure can be applied in the prophylaxis and/or treatment of coronaviral infections by any medically acceptable administration route to a patient in need thereof. Such medically acceptable administration routes can be e.g. by inhalation, by intubation, orally, parenterally, intraperitoneally, intravenously, intraarterially, intramuscularly, topically, transdermally, subcutaneously, intradermally, sublingually, conjunctivally, intravaginally, rectally or nasally.

In another aspect of the invention a pharmaceutical composition for use in the prophylaxis or treatment of a coronaviral infection is disclosed, wherein said composition comprises at least an inhibitor of human deubiquitinases USP7 and/or USP47 or one of its pharmaceutically acceptable salts, hydrates or solvates according to the disclosure, a carrier and at least one pharmaceutically acceptable excipient.

The term “pharmaceutically acceptable excipient(s)” refers to natural or synthetic compounds that are added to a pharmaceutical formulation alongside the pharmaceutical active agent. They may help to bulk up the formulation, to enhance the desired pharmacokinetic properties or the stability of the formulation, as well as being beneficial in the manufacturing process. Advantageous classes of excipients according to the invention include, carriers, binding agents, colorants, buffers, preservatives, antioxidants, coatings, sweeteners, thickening agents, pH-regulators, acidity regulators acidifiers, solvents, isotonizing agents, penetration enhancers, disintegrants, glidants, lubricants, emulsifiers, solubilizing agents, stabilizers, diluents, anti-caking agents (antiadherents), sorbents, foaming agents, anti-foaming agents, opacifiers, fatliquors, consistency enhancers, hydrotropes, aromatic and flavoring substances.

In general, one or more pharmaceutically acceptable carriers are added to a pharmaceutically active agent. Eligible are all carriers known in the art and combinations thereof. In solid dosage forms they can be for example plant and animal fats, waxes, paraffins, starch, tragacanth, cellulose derivatives, polyethylene glycols, silicones, bentonites, silica, talcum, zinc oxide. For liquid dosage forms and emulsions suitable carriers are for example solvents, solubilizing agents, emulsifiers such as water, ethanol, isopropanol, ethyl carbonate, ethyl acetate, benzyl alcohol, benzyl benzoate, propylene glycol, 1,3-butyl glycol, cotton seed oil, peanut oil, olive oil, castor oil, sesame oil, glycerol fatty acid esters, polyethylglycols, fatty acid esters of sorbitan. Suspensions according to the invention may use carriers known in the art such as diluents (e.g. water, ethanol or propylene glycol), ethoxylated isostearyl alcohols, polyoxyethylene and polyoxyethylene sorbitan esters, microcrystalline cellulose, bentonites, agar agar, tragacanth.

The term binding agents refers to substances that bind powders or glue them together, rendering them cohesive through granule formation. They serve as a “glue” of the formulation. Binding agents increase the cohesive strength of the provided diluent or filler.

Suitable binding agents are for example starch from wheat, corn, rice or potato, gelatin, naturally occurring sugars such as glucose, sucrose or beta-lactose, sweeteners from corn, natural and synthetic gums such as acacia, tragacanth or ammonium calcium alginate, sodium alginate, carboxymethyl cellulose, sodium carboxymethyl cellulose, hydroxypropyl carboxymethyl cellulose, polyethylene glycol, polyvinyl pyrrolidone, magnesium aluminum silicate, waxes and others. The percentage of the binding agent in the composition can range from 1-30% by weight, preferred 2-20% by weight, more preferred 3-10% by weight and most preferred 3-6% by weight.

Colorants are excipients that bestow a colorization to the pharmaceutical formulation. These excipients can be food colorants. They can be adsorbed on a suitable adsorption means such as clay or aluminum oxide. A further advantage of a colorant is that it may visualize spilled aqueous solution on the nebulizer and/or the mouthpiece to facilitate cleaning. The amount of the colorant may vary between 0.01 and 10% per weight of the pharmaceutical composition, preferred between 0.05 and 6% per weight, more preferred between 0.1 and 4% per weight, most preferred between 0.1 and 1% per weight.

Suitable pharmaceutical colorants are for example curcumin, riboflavin, riboflavin-5′-phosphate, tartrazine, alkannin, quinolione yellow WS, Fast Yellow AB, riboflavin-5′-sodium phosphate, yellow 2G, Sunset yellow FCF, orange GGN, cochineal, carminic acid, citrus red 2, carmoisine, amaranth, Ponceau 4R, Ponceau SX, Ponceau 6R, erythrosine, red 2G, Allura red AC, Indathrene blue RS, Patent blue V, indigo carmine, Brilliant blue FCF, chlorophylls and chlorophyllins, copper complexes of chlorophylls and chlorophyllins, Green S, Fast Green FCF, Plain caramel, Caustic sulphite caramel, ammonia caramel, sulphite ammonia caramel, Black PN, Carbon black, vegetable carbon, Brown FK, Brown HT, alpha-carotene, beta-carotene, gamma-carotene, annatto, bixin, norbixin, paprika oleoresin, capsanthin, capsorubin, lycopene, beta-apo-8′-carotenal, ethyl ester of beta-apo-8′-carotenic acid, flavoxanthin, lutein, cryptoxanthin, rubixanthin, violaxanthin, rhodoxanthin, canthaxanthin, zeaxanthin, citranaxanthin, astaxanthin, betanin, anthocyanins, saffron, calcium carbonate, titanium dioxide, iron oxides, iron hydroxides, aluminum, silver, gold, pigment rubine, tannin, orcein, ferrous gluconate, ferrous lactate.

Moreover, buffer solutions are preferred for liquid formulations, in particular for pharmaceutical liquid formulations. The terms buffer, buffer system and buffer solution, in particular of an aqueous solution, refer to the capacity of the system to resist a pH change by the addition of an acid or a base, or by dilution with a solvent. Preferred buffer systems may be selected from the group comprising formate, lactate, benzoic acid, oxalate, fumarate, aniline, acetate buffer, citrate buffer, glutamate buffer, phosphate buffer, succinate, pyridine, phthalate, histidine, MES (2-(N-morpholino) ethanesulfonic acid), maleic acid, cacodylate (dimethyl arsenate), carbonic acid, ADA (N-(2-acetamido)imino diacetic acid, PIPES (4-piperazine-bis-ethanesulfonic acid), BIS-TRIS propane (1,3-bis[tris(hydroxymethyl)methylaminol] propane), ethylene diamine, ACES (2-[(amino-2-oxoethyl)amino]ethanesulfonic acid), imidazole, MOPS (3-(N-morphino) propanesulfonic acid), diethyl malonic acid, TES (2-[tris(hydroxymethyl)methyl]aminoethanesulfonic acid), HEPES (N-2-hydroxyethylpiperazine-N′-2-ethanesulfonic acid), as well as other buffers with a pKₐ between 3.8 and 7.7.

Preferred are carbonic acid buffers such as acetate buffer and dicarboxylic acid buffers such as fumarate, tartrate and phthalate as well as tricarboxylic acid buffers such as citrate.

A further group of preferred buffers are inorganic buffers such as sulfate hydroxide, borate hydroxide, carbonate hydroxide, oxalate hydroxide, calcium hydroxide and phosphate buffers. Another group of preferred buffers are nitrogen-containing puffers such as imidazole, diethylene diamine and piperazine. Furthermore preferred are sulfonic acid buffers such as TES, HEPES, ACES, PIPES, [(2-hydroxy-1,1-bis-(hydroxymethyl)ethyl)amino]-1-propanesulfonic acid (TAPS), 4-(2-hydroxyethyl)piperazine-1-propanesulfonic acid (EEPS), MOPS and N,N-bis-(2-hydroxyethyl)-2-aminoethanesulfonic acid (BES). Another group of preferred buffers are glycine, glycyl-glycine, glycyl-glycyl-glycine, N,N-bis-(2-hydroxyethyl)glycine and N-[2-hydroxy-1,1-bis(hydroxymethyl)ethyl]glycine (tricine). Preferred are also amino acid buffers such as glycine, alanine, valine, leucine, isoleucine, serine, threonine, phenylalanine, tyrosine, tryptophan, lysine, arginine, histidine, aspartate, glutamate, asparagine, glutamine, cysteine, methionine, proline, 4-hydroxy proline, N,N,N-trimethyllysine, 3-methyl histidine, 5-hydroxy-lysine, o-phosphoserine, gamma-carboxyglutamate, [epsilon]-N-acetyl lysine, [omega]-N-methyl arginine, citrulline, ornithine and their derivatives.

Preservatives for liquid and/or solid dosage forms can be used on demand. They may be selected from the group comprising, but not limited to, sorbic acid, potassium sorbate, sodium sorbate, calcium sorbate, methyl paraben, ethyl paraben, methyl ethyl paraben, propyl paraben, benzoic acid, sodium benzoate, potassium benzoate, calcium benzoate, heptyl p-hydroxybenzoate, sodium methyl para-hydroxybenzoate, sodium ethyl para-hydroxybenzoate, sodium propyl para-hydroxybenzoate, benzyl alcohol, benzalkonium chloride, phenylethyl alcohols, cresols, cetylpyridinium chloride, chlorbutanol, thiomersal (sodium 2-(ethylmercurithio) benzoic acid), sulfur dioxide, sodium sulfite, sodium bisulfite, sodium metabisulfite, potassium metabisulfite, potassium sulfite, calcium sulfite, calcium hydrogen sulfite, potassium hydrogen sulfite, biphenyl, orthophenyl phenol, sodium orthophenyl phenol, thiabendazole, nisin, natamycin, formic acid, sodium formate, calcium formate, hexamine, formaldehyde, dimethyl dicarbonate, potassium nitrite, sodium nitrite, sodium nitrate, potassium nitrate, acetic acid, potassium acetate, sodium acetate, sodium diacetate, calcium acetate, ammonium acetate, dehydroacetic acid, sodium dehydroacetate, lactic acid, propionic acid, sodium propionate, calcium propionate, potassium propionate, boric acid, sodium tetraborate, carbon dioxide, malic acid, fumaric acid, lysozyme, copper-(II)-sulfate, chlorine, chlorine dioxide and other suitable substances or compositions known to the person skilled in the art.

The addition of a sufficient amount of antioxidants is particularly preferable for liquid and topical dosage forms. Suitable examples for antioxidants include sodium metabisulfite, alpha-tocopherol, ascorbic acid, maleic acid, sodium ascorbate, ascorbyl palmitate, butylated hydroxyanisol, butylated hydroxytoluene, fumaric acid or propyl gallate. Preferred is the use of sodium metabisulfite, alpha-tocopherol and ascorbyl palmitate.

Tablets or pills are usually coated, i.e. the coating constitutes the outer layer. This can be a film coating, a sugar coating with saccharides and a compression coating. Pharmaceutically acceptable varnishes or waxes, HPMC (hydroxypropylmethylcellulose), MC (methylcellulose) or HPC (hydroxypropylcellulose) can be used. Such a coating may help to disguise the taste, to ease the swallowing or the identification. Often plasticizers and pigments are included in the coating. Capsules normally have a gelatinous envelope that encloses the active substance. The specific composition and thickness of this gelatinous layer determines how fast absorption takes place after ingestion of the capsule. Of special interest are sustained release formulations, as known in the art.

Suitable sweeteners can be selected from the group comprising mannitol, glycerol, acesulfame potassium, aspartame, cyclamate, isomalt, isomaltitol, saccharin and its sodium, potassium and calcium salts, sucralose, alitame, thaumatin, glycyrrhizin, neohesperidine dihydrochalcone, steviol glycosides, neotame, aspartame-acesulfame salt, maltitol, maltitol syrup, lactitol, xylitol, erythritol.

Suitable thickening agents can be selected from the group comprising, but not limited to, polyvinyl pyrrolidone, methyl cellulose, hydroxypropyl methyl cellulose, hydroxypropyl cellulose, dextrins, polydextrose, modified starch, alkaline modified starch, bleached starch, oxidized starch, enzyme-treated starch, monostarch phosphate, distarch phosphate esterified with sodium trimetaphosphate or phosphorus oxychloride, phosphate distarch phosphate, acetylated distarch phosphate, starch acetate esterified with acetic anhydride, starch acetate esterified with vinyl acetate, acetylated distarch adipate, acetylated distarch glycerol, distarch glycerin, hydroxypropyl starch, hydroxy propyl distarch glycerin, hydroxypropyl distarch phosphate, hydroxypropyl distarch glycerol, starch sodium octenyl succinate, acetylated oxidized starch, hydroxyethyl cellulose.

Suitable pH-regulators for liquid dosage forms are e.g. sodium hydroxide, hydrochloric acid, buffer substances such as sodium dihydrogen phosphate or disodium hydrogenphosphate.

Suitable acidity regulators can be selected from the group comprising acetic acid, potassium acetate, sodium acetate, sodium diacetate, calcium acetate, carbon dioxide, malic acid, fumaric acid, sodium lactate, potassium lactate, calcium lactate, ammonium lactate, magnesium lactate, citric acid, mono-, di-, trisodium citrate, mono-, di-, tripotassium citrate, mono-, di-, tricalcium citrate, tartaric acid, mono-, disodium tartrate, mono-, dipotassium tartrate, sodium potassium tartrate, ortho-phosphoric acid, lecithin citrate, magnesium citrate, ammonium malate, sodium malate, sodium hydrogen malate, calcium malate, calcium hydrogen malate, adipic acid, sodium adipate, potassium adipate, ammonium adipate, succinic acid, sodium fumarate, potassium fumarate, calcium fumarate, ammonium fumarate, 1,4-heptonolactone, triammonium citrate, ammonium ferric citrate, calcium glycerophosphate, isopropyl citrate, potassium carbonate, potassium bicarbonate, ammonium carbonate, ammonium bicarbonate, magnesium carbonate, magnesium bicarbonate, ferrous carbonate, ammonium sulfate, aluminum potassium sulfate, aluminum ammonium sulfate, sodium hydroxide, potassium hydroxide, ammonium hydroxide, magnesium hydroxide, gluconic acid.

Acidifiers use to be inorganic chemicals that either produce or become acid. Suitable examples are: Ammonium chloride, calcium chloride.

Suitable solvents may be selected from the group comprising, but not limited to, water, carbonated water, water for injection, water with isotonizing agents, saline, isotonic saline, alcohols, particularly ethyl and n-butyl alcohol, and mixtures thereof.

Suitable isotonizing agents are for example pharmaceutically acceptable salts, in particular sodium chloride and potassium chloride, sugars such as glucose or lactose, sugar alcohols such as mannitol and sorbitol, citrate, phosphate, borate and mixtures thereof.

Penetration enhancers (permeation or permeability enhancers) are substances that temporarily diminish the barrier of the skin and promote or accelerate the absorption of cosmetic agents. Suitable penetration enhancers can be selected from the group comprising, but not limited to, dimethyl isosorbide (Arlasolve), dimethyl sulfoxide (DMSO) and its analogues, dimethyl formamide (DMF), azone (1-dodecylazacycloheptan-2-one), pyrrolidones such as 2-pyrrolidone, fatty acids such as oleic acid, lauric acid, myristic acid and capric acid, nonic surfactants such as polyoxyethylene-2-oleyl ether and polyoxyethylene-2-stearyl ether, terpenes, terpenoids and sesquiterpenes such as those from essential oils of eucalyptus, chenopodium and ylang-ylang, oxazolidinones such as 4-decyloxazolidin-2-one, turpentine oil, pine oil, menthol.

Suitable disintegrants can be selected from the group comprising starch, cold water-soluble starches such as carboxymethyl starch, cellulose derivatives such as methyl cellulose and sodium carboxymethyl cellulose, microcrystalline cellulose and cross-linked microcrystalline celluloses such as croscarmellose sodium, natural and synthetic gums such as guar, agar, karaya (Indian tragacanth), locust bean gum, tragacanth, clays such as bentonite, xanthan gum, alginates such as alginic acid and sodium alginate, foaming compositions a.o. Moisture expansion is supported by for example starch, cellulose derivatives, alginates, polysaccharides, dextrans, cross-linked polyvinyl pyrrolidone. The amount of the disintegrant in the composition may vary between 1 and 40% per weight, preferred between 3 and 20% per weight, most preferred between 5 and 10% per weight.

Glidants are materials that prevent a baking of the respective supplements and improve the flow characteristics of granulations so that the flow is smooth and constant. Suitable glidants comprise silicon dioxide, magnesium stearate, sodium stearate, starch and talcum. The amount of the glidant in the composition may vary between 0.01 and 10% per weight, preferred between 0.1 and 7% per weight, more preferred between 0.2 and 5% per weight, most preferred between 0.5 and 2% per weight.

The term “lubricants” refers to substances that are added to the dosage form in order to facilitate tablets, granulates etc. to be released from the press mold or the outlet nozzle. They diminish friction or abrasion. Lubricants are usually added shortly before pressing, as they should be present on the surface of the granules and between them and the parts of the press mold. The amount of the lubricant in the composition may vary between 0.05 and 15% per weight, preferred between 0.2 and 5% per weight, more preferred between 0.3 and 3% per weight, most preferred between 0.3 and 1.5% per weight. Suitable lubricants are a.o. sodium oleate, metal stearates such as sodium stearate, calcium stearate, potassium stearate and magnesium stearate, stearic acid, sodium benzoate, sodium acetate, sodium chloride, boric acid, waxes having a high melting point, polyethylene glycol.

Emulsifiers can be selected for example from the following anionic and non-ionic emulsifiers: Anionic emulsifier waxes, cetyl alcohol, cetylstearyl alcohol, stearic acid, oleic acid, polyoxyethylene polyoxypropylene block polymers, addition products of 2 to 60 mol ethylene oxide to castor oil and/or hardened castor oil, wool wax oil (lanolin), sorbitan esters, polyoxyethylene alkyl esters, polyoxyethylene sorbitan fatty acid esters, polyoxyethene sorbitan monolaurate, polyoxyethene sorbitan monooleate, polyoxyethene sorbitan monopalmitate, polyoxyethene sorbitan monostearate, polyoxyethene sorbitan tristearate, polyoxyethene stearate, polyvinyl alcohol, metatartaric acid, calcium tartrate, alginic acid, sodium alginate, potassium alginate, ammonium alginate, calcium alginate, propane-1,2-diol alginate, carrageenan, processed eucheuma seaweed, locust bean gum, tragacanth, acacia gum, karaya gum, gellan gum, gum ghatti, glucomannane, pectin, amidated pectin, ammonium phosphatides, brominated vegetable oil, sucrose acetate isobutyrate, glycerol esters of wood rosins, disodium phosphate, trisodium diphosphate, tetrasodium diphosphate, dicalcium diphosphate, calcium dihydrogen diphosphate, sodium triphosphate, pentapotassium triphosphate, sodium polyphosphates, sodium calcium polyphosphate, calcium polyphosphates, ammonium polyphosphate, beta-cyclodextrin, powdered cellulose, methyl cellulose, ethyl cellulose, hydroxypropyl cellulose, hydroxypropyl methylcellulose, ethyl methyl cellulose, carboxymethyl cellulose, sodium carboxymethyl cellulose, ethyl hydroxyethyl cellulose, croscarmellose, enzymically hydrolyzed carboxymethyl cellulose, mono- and diglycerides of fatty acids, glyceryl monostearate, glyceryl distearate, acetic acid esters of mono- and diglycerides of fatty acids, lactic acid esters of mono- and diglycerides of fatty acids, citric acid esters of mono- and diglycerides of fatty acids, tartaric acid esters of mono- and diglycerides of fatty acids, mono- and diacetyl tartaric acid esters of mono- and diglycerides of fatty acids, mixed acetic and tartaric acid esters of mono- and diglycerides of fatty acids, succinylated monoglycerides, sucrose esters of fatty acids, sucroglycerides, polyglycerol esters of fatty acids, polyglycerol polyricinoleate, propane-1,2-diol esters of fatty acids, propylene glycol esters of fatty acids, lactylated fatty acid esters of glycerol and propane-1, thermally oxidized soy bean oil interacted with mono- and diglycerides of fatty acids, dioctyl sodium sulphosuccinate, sodium stearoyl-2-lactylate, calcium stearoyl-2-lactylate, stearyl tartrate, stearyl citrate, sodium stearoyl fumarate, calcium stearoyl fumarate, stearyl tartrate, stearyl citrate, sodium stearoyl fumarate, calcium stearoyl fumarate, sodium laurylsulfate, ethoxylated mono- and diglycerides, methyl glucoside-coconut oil ester, sorbitan monostearate, sorbitan tristrearate, sorbitan monolaurate, sorbitan monooleate, sorbitan monopalmitate, sorbitan trioleate, calcium sodium polyphosphate, calcium polyphosphate, ammonium polyphosphate, cholic acid, choline salts, distarch glycerol, starch sodium octenyl succinate, acetylated oxidized starch. Preferred are glycerin monooleate, stearic acid, phospholipids such as lecithin.

Suitable as surface-active solubilizing agents (solubilizers) are for example diethylene glycol monoethyl ester, polyethyl propylene glycol co-polymers, cyclodextrins such as α- and β-cyclodextrin, glyceryl monostearates such as Solutol HS 15 (Macrogol-15-hydroxystearate from BASF, PEG 660-15 hydroxystearates), sorbitan esters, polyoxyethylene glycol, polyoxyethylene sorbitanic acid esters, polyoxyethylene sorbitan monooleate, polyoxyethylene oxystearic acid triglyceride, polyvinyl alcohol, sodium dodecyl sulfate, (anionic) glyceryl monooleates.

Stabilizers are substances that can be added to prevent unwanted changes. Though stabilizers are not real emulsifiers they may also contribute to the stability of emulsions. Suitable examples for stabilizers are oxystearin, xanthan gum, agar, oat gum, guar gum, tara gum, polyoxyethene stearate, aspartame-acesulfame salt, amylase, proteases, papain, bromelain, ficin, invertase, polydextrose, polyvinyl pyrrolidone, polyvinyl polypyrrolidone, triethyl citrate, maltitol, maltitol syrup.

Diluents or fillers are inactive substances added to drugs in order to handle minimal amounts of active agents. Examples for suitable diluents are water, mannitol, pre-gelatinized starch, starch, microcrystalline cellulose, powdered cellulose, silicified microcrystalline cellulose, dibasic calcium phosphate dihydrate, calcium phosphate, calcium carbonate, hydroxypropyl cellulose, hydroxyethyl cellulose, hydroxypropyl methylcellulose, polyethylene glycol, xanthum gum, gum arabic or any combination thereof.

Anti-caking agents (antiadherents) can be added to a supplement or a composition of supplements in order to prevent the formation of lumps and for easing packaging, transport, release from the at least one chamber of the dispensing cap and consumption. Suitable examples include tricalcium phosphate, powdered cellulose, magnesium stearate, sodium bicarbonate, sodium ferrocyanide, potassium ferrocyanide, calcium ferrocyanide, bone phosphate, sodium silicate, silicon dioxide, calcium silicate, magnesium trisilicate, talcum powder, sodium aluminosilicate, potassium aluminum silicate, calcium aluminosilicate, bentonite, aluminum silicate, stearic acid, polydimethyl siloxane.

Sorbents are materials that soak up oil from the water. Suitable examples include natural sorbents such as peat moss, sawdust, feathers, and anything else natural that contains carbon and synthetic sorbents such as polyethylene and nylon. Sorbents are used for tablet/capsule moisture-proofing by limited fluid sorbing (taking up of a liquid or a gas either by adsorption or by adsorption) in a dry state.

In some galenic formulations it may be desirable that a liquid oral dosage form generates some foam on being dissolved. Such an effect can be supported through the addition of a foaming agent that reduces the surface tension of the liquid, thus facilitating the formation of bubbles, or it increases its colloidal stability by inhibiting coalescence of bubbles. Alternatively, it may stabilize foam. Suitable examples include mineral oil, quillaia extract, triethyl citrate, sodium lauryl ether sulfate, sodium lauryl sulfate, ammonium lauryl sulfate.

Alternatively, some liquid oral dosage forms may appear slightly foamy upon preparation. Though this does not interfere with the desired application it may affect patient compliance in case of a medication or the commercial success in case of dietary supplements. Therefore, it may be desirable to add a pharmaceutically acceptable anti-foaming agent (defoamer). Examples are polydimethylsiloxane or silicone oil in dietary supplements or simethicone in pharmaceuticals.

Opacifiers are substances that render the liquid dosage for, opaque, if desired. They must have a refractive index substantially different from the solvent, in most cases here water. At the same time, they should be inert to the other components of the composition. Suitable examples include titanium dioxide, talc, calcium carbonate, behenic acid, cetyl alcohol, or mixtures thereof.

Suitable fatliquors are e.g. oleic acid decyl ester, hydrated castor oil, light mineral oil, mineral oil, polyethylene glycol, sodium laurylsulfate.

Consistency enhancers are e.g. cetyl alcohol, cetyl ester wax, hydrated castor oil, microcrystalline waxes, non-ionic emulsifier waxes, beeswax, paraffin or stearyl alcohol.

Suitable hydrotropes are alcohols such as ethanol, isopropyl alcohol or polyols such as glycerin.

Suitable aromatic and flavoring substances comprise above all essential oils that can be used for this purpose. In general, this term refers to volatile extracts from plants or parts of plants with the respective characteristic smell. They can be extracted from plants or parts of plants by steam distillation.

Suitable examples are: Essential oils, respectively aromatic substances from sage, cloves, chamomile, anise, star anise, thyme, tea tree, peppermint, mint oil, menthol, cineol, borneol, zingerol, eucalyptus oil, mango, figs, lavender oil, chamomile blossoms, pine needles, cypress, oranges, rosewood, plum, currant, cherry, birch leaves, cinnamon, limes, grapefruit, tangerine, juniper, valerian, lemon balm, lemon grass, palmarosa, cranberry, pomegranate, rosemary, ginger, pineapple, guava, echinacea, ivy leave extract, blueberry, kaki, melons etc. or mixtures thereof, as well as mixtures of menthol, peppermint and star anise oil or menthol and cherry flavor.

These aromatic or flavoring substances can be included in the range of 0.0001 to 10% per weight (particularly in a composition), preferred 0.001 to 6% per weight, more preferred 0.001 to 4% per weight, most preferred 0.01 to 1% per weight, with regard to the total composition. Application- or single case-related it may be advantageous to use differing quantities.

According to the disclosure all of the aforementioned excipients and classes of excipients can be used without limitation alone or in any conceivable combination thereof, as long as the inventive use is not thwarted, toxic actions may occur, or respective national legislations are infracted.

In another aspect of the invention the present application relates to a deubiquitinase inhibitor according to the disclosure or to a combination according to the disclosure for use in the prophylaxis or treatment of a coronaviral infection in a formulation for oral administration.

It refers likewise to a pharmaceutical composition for use as described before, wherein the pharmaceutical composition is a formulation for oral administration.

In another aspect of the invention the present disclosure relates to a deubiquitinase inhibitor according to the disclosure or to a combination according to the disclosure for use in the prophylaxis or treatment of a coronaviral infection in a formulation for inhalatory administration.

It relates likewise to a pharmaceutical composition for use as described before, wherein the pharmaceutical composition is a formulation for inhalatory administration.

For an effective prophylactic or therapeutic treatment of coronavirus infections that may cause pneumonia, pulmonary edema and/or acute lung injury it is advantageous that the at least one deubiquitinase inhibitor according to the disclosure reaches the patient's alveoli. Therefore, the particle size must be sufficiently small to reach the lowest parts of the airways of the pulmonary tissue. The best inhalatory device class for inhalatory application of a pharmaceutically active agent are the so-called mesh nebulizers described before. In the scope of the present application practically all mesh nebulizers known in the art can be used, from rather simple single-use mesh nebulizers for cough and cold or for fancy purposes to sophisticated high-end mesh nebulizers for clinical or domestic treatment of serious diseases or conditions of the lower airways.

Suitable commercially available mesh nebulizers, jet nebulizers, ultrasonic nebulizers, dry powder inhalers and (pressurized) metered-dose inhalers comprise, without being limiting, eFlow® rapid, PARI LC STAR®, PARI Velox and PARI Velox Junior (PARI GmbH, Starnberg, Germany), Philips Respironics I-neb and Philips InnoSpire Go (Koninklijke Philips N.V., Eindhoven, Netherlands), VENTA-NEB®-ir, OPTI-NEB®, M-neb® dose mesh nebulizer inhalation MN-300/8, M-Neb Flow+ and M-neb® mesh nebulizer MN-300/X (NEBU-TEC, Eisenfeld, Germany), Hcmed Deepro HCM-86C and HCM860 (HCmed Innovations Co., Ltd, Taipei, Taiwan), OMRON MicroAir U22 and U100 (OMRON, Kyoto, Japan), Aerogen® Solo, Aerogen® Ultra and Aerogen® PRO (Aerogen, Galway, Ireland), KTMED NePlus NE-SM1 (KTMED Inc., Seoul, South Korea), Vectura Bayer Breelib™ (Bayer AG, Leverkusen, Germany), Vectura Fox, MPV Truma and MicroDrop® Smarty (MPV MEDICAL GmbH, Kirchheim, Germany), MOBI MESH (APEX Medical, New Taipei City, Taiwan), B.Well WN-114, TH-134 and TH-135 (B.Well Swiss AG, Widnau, Switzerland), Babybelle Asia BBU01 (Babybelle Asia Ltd., Hongkong), CA-MI Kiwi and others (CA-MI sri, Langhirano, Italy), Diagnosis PRO MESH (Diagnosis S.A., Bialystok, Poland), DIGI O₂ (DigiO₂ International Co., Ltd., New Taipei City, Taiwan), feellife AIR PLUS, AEROCENTRE+, AIR 360+, AIR GARDEN, AIRICU, AIR MASK, AIRGEL BOY, AIR ANGEL, AIRGEL GIRL and AIR PRO 4 (Feellife Health Inc., Shenzhen, China), Hannox MA-02 (Hannox International Corp., Taipei, Taiwan), Health and Life HL100 and HL100A (HEALTH & LIFE Co., Ltd., New Taipei City, Taiwan), Honsun NB-810B (Honsun Co., Ltd., Nantong, China), K-jump® KN-9100 (K-jump Health Co., Ltd., New Taipei City, Taiwan), microlife NEB-800 (Microlife AG, Widnau, Switzerland), OK Biotech Docspray (OK Biotech Co., Ltd., Hsinchu City, Taiwan), Prodigy Mini-Mist® (Prodigy Diabetes Care, LLC, Charlotte, USA), Quatek NM211, NE203, NE320 and NE403 (Big Eagle Holding Ltd., Taipei, Taiwan), Simzo NBM-1 and NBM-2 (Simzo Electronic Technology Ltd., Dongguan, China), Mexus® BBU01 and BBU02 (Tai Yu International Manufactory Ltd., Dongguan, China), TaiDoc TD-7001 (TaiDoc Technology Co., New Taipei City, Taiwan), Vibralung® and HIFLO Miniheart Circulaire II (Westmed Medical Group, Purchase, USA), KEJIAN (Xuzhou Kejian Hi-Tech Co., Ltd., Xuzhou, China), YM-252, P&S-T45 and P&S-360 (TEKCELEO, Valbonne, France), Maxwell YS-31 (Maxwell India, Jaipur, India), Kernmed® JLN-MB001 (Kernmed, Durmersheim, Germany).

Preferred are mesh nebulizers with a piezoelectric activation of the nebulization process, respectively vibrating mesh nebulizers.

Mesh nebulizers can be classified into two groups according to patient interaction: Continuous mode devices and trigger-activated devices. In continuous mode mesh nebulizers the nebulized aerosol is continuously released into the mouth piece and the patient has to inhale the provided aerosol. In trigger-activated devices a defined amount of aerosol is released only upon an active and deep inspiratory breath. This way a far larger amount of active agent-containing aerosol is inhaled and reaches the lowest airways than with continuous mode devices. The latter lose a large amount of active agent-containing aerosol either to the surrounding or on the passage of the upper airways, as the aerosol release is not coupled to the respiratory cycle.

Therefore, trigger-activated mesh nebulizers are preferred, in particular vibrating mesh nebulizers.

Particularly preferred are trigger-activated mesh nebulizers with a piezoelectric activation of the nebulization process.

Preferred are the mesh nebulizer models PARI eFlow® rapid, Philips Respironics I-neb, Philips InnoSpire Go, M-neb® dose⁺ mesh nebulizer inhalation MN-300/8, Hcmed Deepro HCM-86C and HCM860, OMRON MicroAir U100, Aerogen® Solo, KTMED NePlus NE-SM1, Vectura Fox, Vectura Bayer Breelib™.

The most preferred vibrating mesh nebulizer models are high-end models such as PARI eFlow® rapid, PARI Velox, Philips Respironics I-neb, M-neb® dose⁺ mesh nebulizer inhalation MN-300/8, Aerogen® Solo, Vectura Fox, Vectura Bayer Breelib™.

Thus, in another aspect of the invention the present disclosure relates to a deubiquitinase inhibitor according to the disclosure or of a combination according to the disclosure for use in the prophylaxis or treatment of a coronaviral infection in a formulation for inhalatory administration, wherein the inhalatory administration is carried out by means of a vibrating mesh nebulizer.

It relates also to a pharmaceutical composition for use for inhalatory administration as described before, wherein the inhalatory administration is carried out by means of a vibrating mesh nebulizer.

The mean droplet size is usually characterized as MMAD (median mass aerodynamic diameter). The individual droplet size is referred to as MAD (mass aerodynamic diameter). This value indicates the diameter of the nebulized particles (droplets) at which 50% are smaller or larger, respectively. Particles with a MMAD>10 μm normally do not reach the lower airways, they often get stuck in the throat. Particles with a MMAD>5 μm and <10 μm usually reach the bronchi but not the alveoli. Particles between 100 nm and 1 μm MMAD don't deposit in the alveoli and are exhaled immediately. Therefore, the optimal range is between 1 μm and 5 μm MMAD. Recent publications even favor a narrower range between 3.0 μm and 4.0 μm (cf. Amirav et al. (2010) J Allergy Clin Immunol 25: 1206-1211; Haidl et al. (2012) Pneumologie 66: 356-360).

A further commonly accepted quality parameter is the percentage of the particles in the generated aerosol with a diameter in the range of 1 μm to 5 μm (FPM; fine particle mass). FPM is a measure for the particle distribution. It is calculated by subtracting the percentage of the particles in the generated aerosol with a diameter in the range <1 μm from the overall percentage of the particles in the generated aerosol with a diameter in the range <5 μm (FPF; fine particle fraction).

In another aspect of the invention the present application refers also to a method for producing an aerosol according to the invention, comprising the following steps:
a) filling 0.1 ml to 5 ml of an aqueous solution containing the at least one deubiquitinase inhibitor according to the disclosure and optionally at least one pharmaceutically acceptable excipient into the nebulization chamber of a mesh nebulizer,
b) starting vibration of the mesh of the mesh nebulizer at a frequency of 80 kHz to 200 kHz, and
c) discharging the generated aerosol at the side of the mesh of the mesh nebulizer opposite to the nebulization chamber.

The vibration frequency of vibrating mesh nebulizers is normally in the range of 80 kHz to 200 kHz, preferred 90 kHz to 180 kHz, more preferred 100 kHz to 160 kHz, most preferred 105 kHz to 130 kHz (cf. Chen, The Aerosol Society. DDL2019; Gardenshire et al. (2017) A Guide to Aerosol Delivery Devices for Respiratory Therapists, 4th ed.).

Thus, the aforementioned method is also disclosed with said vibration frequency ranges.

The method according to the invention is thus characterized in that at least 80% in weight, preferred at least 85% in weight, most preferred at least 90% in weight of the at least one deubiquitinase inhibitor according to the disclosure contained in said aqueous solution are nebulized in the generated aerosol.

The method of the invention is particularly effective in nebulizing a high percentage of the pharmaceutically active agent(s) from the provided aqueous solution during a short time. This is an important feature for patient compliance. A considerable percentage of the patient population finds the inhalatory process to be uncomfortable, weary and physically demanding. On the other hand, the patient's active cooperation is essential for an effective and targeted inhalatory application. Therefore, it is desirable that a therapeutically sufficient amount is applied during a period of time as short as possible. Surprisingly, it showed that during a three minutes time span 95% of the substance provided in the aqueous solution could be nebulized. This is an ideal time span for a high patient compliance.

Therefore, the method according to the invention is thus characterized in that at least 80% of the generated aerosol are produced during three minutes after starting nebulization in the mesh nebulizer, preferred at least 85% and most preferred at least 90%.

While the pharmaceutically active agent is usually provided in a single dosage container for every nebulization procedure the nebulizer and/or the mouthpiece can be used over a certain period of time and have to be replaced at certain intervals. A cleaning of the nebulizer and the mouthpiece is recommended by default after each nebulization. But herein patient compliance cannot be reasonably taken for granted. But even after a meticulous cleaning there are always some deposits of the aerosol in the nebulization chamber, the outlet and/or the mouthpiece. As the aerosol is produced from an aqueous solution these depositions bear the risk of producing a bioburden of bacteria that might contaminate the inhaled aerosol. Deposits may also plug holes in the mesh membrane of the mesh nebulizer. In general, the nebulizer and/or the mouthpiece should be exchanged every one or two weeks. Therefore, it is convenient to offer the medication and the nebulizer as a combined product.

Thus, in another aspect of the invention the present application refers also to a kit comprising a mesh nebulizer and a pharmaceutically acceptable container with an aqueous solution containing the at least one deubiquitinase inhibitor according to the disclosure and optionally at least one pharmaceutically acceptable excipient.

In an alternative kit the at least one deubiquitinase inhibitor according to the disclosure is not provided in form of an aqueous solution but in two separated containers, one for a solid form for the active agent and the other for an aqueous solution. The final aqueous solution is freshly prepared by solving the active agent in the final solution. Thereupon the final aqueous solution is filled into the nebulization chamber of the mesh nebulizer. These two containers can be completely separated containers, e.g. two vials, or e.g. a dual-chamber vial. For solving the active agent e.g. a membrane between the two chambers is perforated to allow for mixing of the content of both chambers.

Thus, the present application discloses also a kit, comprising a mesh nebulizer, a first pharmaceutically acceptable container with water for injection or physiological saline solution and a second pharmaceutically acceptable container with a solid form of the at least one deubiquitinase inhibitor according to the disclosure, wherein optionally at least one pharmaceutically acceptable excipient is contained in the first pharmaceutically acceptable container and/or the second pharmaceutically acceptable container.

The aerosol generated by the method according to the invention is administered, respectively self-administered by means of a mouthpiece. Optionally, such a mouthpiece can be additionally included in the beforementioned kits.

A common way to transfer the provided aqueous solution or final aqueous solution into the nebulization chamber of the mesh nebulizer by means of a syringe equipped with an injection needle. First, the aqueous solution is drawn up into the syringe and then injected into the nebulization chamber. Optionally, such a syringe and/or injection needle can be additionally included in the beforementioned kits. Without being limiting, typical syringes made of polyethylene, polypropylene or cyclic olefin co-polymers can be used, and a typical gauge for a stainless steel injection needle would be in the range of 14 to 27.

In yet another aspect of the invention the present application relates to a deubiquitinase inhibitor according to the disclosure or of a combination according to the disclosure for use in the prophylaxis or treatment of a coronaviral infection in a formulation for sublingual tablets.

It also relates to a pharmaceutical composition for use as described before, wherein the pharmaceutical composition is a formulation for sublingual tablets.

In yet another aspect of the invention the present disclosure relates to a deubiquitinase inhibitor according to the disclosure or of a combination according to the disclosure for use in the prophylaxis or treatment of a coronaviral infection in a liquid dosage form.

The present disclosure relates likewise to a pharmaceutical composition for use as described before, wherein the pharmaceutical composition is a liquid dosage form.

In general, an aqueous solution or a physiological saline solution is preferred. In case of a poorly soluble pharmaceutical agent according to the invention also ethanol or ethanol/water mixtures can be used.

Suitable liquid dosage forms include drops, eyedrops, eardrops or injection solutions.

While SARS-CoV and MERS-CoV infect above all the lower airways SARS-CoV-2 infects first the pharynx/throat area. Only a minor percentage of these patients develops later a pulmonary infection and a pneumonia. While these pharyngeal infections cause usually only mild symptoms as in a cold or no symptoms at all these patients are highly infectious for their environment. In most cases they are unaware that they have become spreaders of the infection. Therefore, there is a medical need to treat coronaviral infections already when they are still in the pharyngeal stage, not only for treating such a patient but also for epidemiologic reasons to prevent the spreading of the epidemic. For patients with a pharyngeal infection only a systemic route of administration, e.g. intravenously or perorally, with a highly effective drug or drug combination that may also cause adverse side effects is not ideal. Thus, it is desirable to provide routes of administration that treat the infected pharyngeal tissue locally.

Therefore, in yet another aspect of the invention the present application relates to a deubiquitinase inhibitor according to the disclosure or of a combination according to the disclosure for use in the prophylaxis or treatment of a coronaviral infection in a formulation for pharyngeal administration.

Administration of a medication to the pharynx can be effected by topical administrations, such as brushing of the throat/pharynx area with a suitable liquid dosage form as drops, a lotion or a tincture, or with a viscous dosage form such as a gel or hydrogel, gurgling with a mouthwash, a sublingual tablet, a lozenge, a throat spray or a posterior pharyngeal wall injection.

A lotion is a low-viscosity topical preparation intended for application to the skin or the mucosa. Lotions are applied to the skin or mucosa with bare hands, a brush, a clean cloth, or cotton wool.

An advantage of a lotion is that it may be spread thinly and may cover a large area of skin or mucosa. Typical drugs that can be administered in form of a lotion include antibiotics, antiseptics, antifungals, corticosteroids, anti-acne agents, soothing, smoothing, moisturizing or protective agents, or anti-allergens.

Most lotions are oil-in-water emulsions using a substance such as cetearyl alcohol to keep the emulsion together, but water-in-oil lotions are also formulated. The key components are the aqueous and oily phases, an emulgent to prevent separation of these two phases and the drug substance(s). A wide variety of excipients such as fragrances, glycerol, petroleum jelly, dyes, preservatives, proteins and stabilizing agents are commonly added to lotions.

Thickness, consistency and viscosity of the lotion can be adjusted during manufacturing. Manufacturing lotions can be carried out in two cycles: a) Emollients and lubricants are dispersed in oil with blending and thickening agents. b) Perfume, color and preservatives are dispersed in the water phase. Pharmaceutically active principles are broken up in both cycles depending on the raw materials involved and the desired properties of the lotion.

A tincture is typically an alcoholic extract or formulation. Solvent concentrations of 25-60% (or even 90%) are common. Other solvents for producing tinctures include vinegar, glycerin, diethyl ether and propylene glycol. Ethanol has the advantage of being an excellent solvent for both acidic and alkaline constituents. A tincture using glycerin is called a glycerite. Glycerin is generally a poorer solvent than ethanol. Vinegar, being acidic, is a better solvent for obtaining alkaloids but a poorer solvent for acidic components.

A gel is a colloid in which the solid disperse phase forms a network in combination with that of the fluid continuous phase, resulting in a viscous semirigid sol. Gel properties range from soft and weak to hard and tough. They are defined as a substantially dilute cross-linked system, which exhibits no flow in the steady-state. By weight, gels are mostly liquid, yet they behave like solids due to a three-dimensional cross-linked network within the liquid. It is the crosslinking within the fluid that gives a gel its consistency and contributes to the adhesive stick. Gels are a dispersion of molecules of a liquid within a solid medium.

A hydrogel is a network of polymer chains that are hydrophilic, sometimes found as a colloidal gel in which water is the dispersion medium. A three-dimensional solid results from the hydrophilic polymer chains being held together by cross-links. Because of the inherent cross-links, the structural integrity of the hydrogel network does not dissolve from the high concentration of water. Hydrogels are highly absorbent (they can contain over 90% water) natural or synthetic polymeric networks. Hydrogels also possess a degree of flexibility very similar to natural tissue, due to their significant water content. In medicine, hydrogels can encapsulate chemical systems which upon stimulation by external factors such as a change of pH may cause specific pharmaceutically active agent(s) to be liberated to the environment, in most cases by a gel-sol transition to the liquid state.

Suitable gel formers can be selected from the group comprising, but not limited to, agar, algin, alginic acid, bentonite, carbomer, carrageenan, hectorite, hydroxyethyl cellulose, hydroxypropyl cellulose, polyvinyl alcohol, polyvinyl pyrrolidone, sodium carbomer.

A mouthwash is a liquid which is held in the mouth passively or swilled around the mouth by contraction of the perioral muscles and/or movement of the head, and may be gargled, where the head is tilted back and the liquid bubbled at the back of the mouth. An aqueous or alcoholic solution of a deubiquitinase inhibitor according to the disclosure can thus be formulated and administered to the pharynx.

Sublingual drug delivery can be an alternative when compared to oral drug delivery as sublingually administered dosage forms bypass hepatic metabolism. A rapid onset of pharmacological effect is often desired for some drugs, especially those used in the treatment of acute disorders. Sublingual tablets disintegrate rapidly, and the small amount of saliva present is usually sufficient for achieving disintegration of the dosage form coupled with better dissolution and increased bioavailability.

The drug must be lipophilic enough to be able to partition through the lipid bilayer, but not so lipophilic such that once it is in the lipid bilayer, it will not partition out again. According to the diffusive model of absorption, the flux across the lipid bilayer is directly proportional to the concentration gradient. Therefore, lower salivary solubility results in lower absorption rates and vice versa. In general, a drug which has been formulated for sublingual should ideally have a molecular weight of less than 500 to facilitate its diffusion. The oral cavity has a narrow pH range which lies between 5.0 to 7.0. The inclusion of a suitable buffer during the formulation of an ionizable drug makes it possible to control the pH of aqueous saliva.

In order to avoid a possibly unpleasant taste or smell of the drug taste masking is needed. Sweeteners, flavors, and other taste-masking agents are essential components. Sugar-based excipients quickly dissolve in saliva and produce endothermic heat of dissolution.

They create a pleasant feeling in the mouth and are most suitable for sublingual tablets along with other flavors.

Typical techniques for manufacturing sublingual tablets include direct compression, compression molding, freeze drying and hot melt extrusion (Khan et al. (2017) J Pharmaceut Res 16: 257-267).

When swallowing is avoided, an administration of a pharmaceutically active agent by means of a sublingual tablet can also reach the pharynx/throat topically. Absorption of the pharmaceutically active agent occurs to a good part via the pharyngeal mucosa.

A lozenge (troche) is a small, disc-shaped or rhombic body composed of solidifying paste containing an astringent, antiseptic, or demulcent drug, used for local treatment of the mouth or throat, the lozenge being held in the mouth until dissolved. The vehicle or base of the lozenge is usually sugar, made adhesive by admixture with acacia or tragacanth, fruit paste, made from black or red currants, confection of rose, or balsam of tolu.

In particular, the present application relates to a deubiquitinase inhibitor according to the disclosure or of a combination according to the disclosure for use in the prophylaxis or treatment of a coronaviral infection in a formulation for pharyngeal administration, wherein the pharyngeal administration is carried out by means of a throat spray.

A throat spray is a medicated liquid administered to the throat as a spray, typically for the treatment of a sore throat or cough.

A throat spray may typically contain a local anesthetic (e.g. lidocaine, benzocaine), an antiseptic (e.g. chlorhexidine, cetylpyridinium chloride), herbal extracts or a combination thereof. Whatever the formulation, it should not contain too much sugar or ethanol, which further irritates the mucosa. And finally, the user should not experience any unpleasant aftertaste.

The standard for throat sprays is currently a metering pump attached to a bottle containing between 10 to 30 ml of a liquid formulation. The formulation is filled into a glass or plastic bottle with the pump fixed by a screw closure, crimped on or simply snapped onto the bottle neck. Irrespective of the fixing option selected, the system should be tight, with no leakage observed during carrying or handling by the user. Usually, the container is made from glass or plastic.

Typically, a throat spray pump will deliver a dose in the range of 50 to 200 μl per actuation. For a targeted administration, the pump will be equipped with an actuator with a prolonged nozzle. The nozzle length may range from 30 to 70 mm. It is easier to target the affected area with such a long-fixed nozzle, but this can be too bulky for users to carry, which is why actuators with foldable or swivel-mounted nozzles are preferred.

Alternatively, devices utilize continuous valves. A continuous valve delivers a targeted treatment but not precise dosing, as the formulation will be aerosolized while the actuator is pressed down. One technical solution is a tin or aluminum can with pressurized head space. When actuating the valve, the elevated internal pressure will force the formulation out of the can as long as the valve stem is pressed down.

A related but more sophisticated system is the bag-on-valve (BOV) system. The product is placed inside a bag while a propellant (in most cases compressed air) is filled in the space between the bag and the outer can. The product is squeezed out of the bag by the compressed air when the continuous valve is actuated. A BOV system will work with any 360° orientation.

Care should be taken, as throat spray formulations may contain ingredients that are very aggressive and can lower the surface tension. A simple test for spray performance will ensure that the formulation can be aerosolized by the system and that the delivered spray pattern and particle size is appropriate for the intended use.

Spray pattern and droplet size distribution are the most important parameters for a throat spray. Spray pattern is a term used to describe the spray angle and the shape of the plume for a fully developed spray. The droplet size is characterized once the spray is fully developed using a laser diffraction method. Fine particles (droplets with less than 10 μm mean dynamic diameter) should be as low as possible to avoid droplet deposition in the lower airways.

Recently, some carragelose-based throat sprays emerged, claiming protection to virus born upper respiratory infections. The first polymer of this platform is Carragelose®, a broadly active anti-viral compound for treating respiratory diseases. The compound prevents the binding of viruses on the mucosal cells, in addition to its moistening effect.

Alternatively, a portable nebulizer with a high output rate and a tuned droplet size for deposition in the upper airways can be used. Breathing through a face mask can deposit droplets on the mucosa of the whole upper airways (cf. Marx and Nadler (2018) Drug Development & Delivery).

In particular, the present application relates to a deubiquitinase inhibitor according to the disclosure or of a combination according to the disclosure for use in the prophylaxis or treatment of a coronaviral infection in a formulation for pharyngeal administration, wherein the pharyngeal administration is carried out by means of a posterior pharyngeal wall injection.

This technique is used for pharyngoplasty by injection of calcium hydroxylapatite and other methods in plastic surgery. However, also a local injection can be made into the pharyngeal tissue in order to administer a pharmaceutically active agent. The injection solution can be roughly the same as for intravenous or intramuscular injections. Preferred are aqueous solutions, physiological saline solutions or, in case of a rather lipophilic pharmaceutically active agent, an ethanol/water mixture.

In a further aspect of the invention the present application relates to a deubiquitinase inhibitor according to the disclosure for use in the prophylaxis or treatment of a coronaviral infection in a formulation for nasal administration.

In particular, the nasal administration is carried out by means of a nasal spray or nose drops.

The common formulation types used for nasal spray products are solutions, suspensions, and emulsions. Nasal spray formulations may be aqueous, hydroalcoholic, or nonaqueous-based. Depending on the type of system, the formulation will include a range of functional excipients, including solvents and cosolvents; mucoadhesive agents; pH buffers; antioxidants; preservatives; osmolality and tonicity agents; penetration enhancers; suspending agents; and surfactants. The choice of formulation type and the excipients selected will be driven by the solubility and stability of the respective deubiquitinase inhibitor according to the disclosure, as well as the concentration needed to deliver an efficacious dose in a typical 100 μl spray (cf. Kulkarni and Shaw (2016) in: Essential Chemistry for Formulators of Semisolid and Liquid Dosages, Elsevier). The aforementioned Carragelose® technique is used also for nasal sprays.

Nose drops are administered in a similar formulation but dropwise instead of a push on the dispenser.

In particular, the present application relates to a deubiquitinase inhibitor according to the disclosure for use in a formulation in the prophylaxis or treatment of a coronaviral infection for nasal administration, wherein the nasal administration is carried out by means of a nasal spray or nose drops.

It is known that the eye mucosae are another entry point of SARS-CoV-2 to the organism, e.g. a person carries the viruses on his hands while rubbing his eyes.

Therefore, the present application relates also to a deubiquitinase inhibitor according to the disclosure, wherein the deubiquitinase inhibitor according to the disclosure is provided in a formulation of eye drops.

Eye drops are mostly aqueous solutions containing a pharmaceutically active agent. The pH is usually adjusted to 7.1 to 7.5. Common buffers for eye drops are boric acid and monobasic sodium phosphate. The tonicity should be adjusted by 0.9% saline (or another isotonizing agent such as potassium nitrate, boric acid, sodium acetate, sodium acetate phosphate buffer or mannitol) to an osmotic pressure isotonic to the cornea epithelium (225-430 mosm/kg). Suitable preservatives include thiomersal, organic mercury compounds such as phenylmercury, benzalkonium chloride, chlorhexidine and benzylic alcohol. For prolonging the contact time viscosity-increasing substances (thickening agents) such as cellulose derivatives (hypromellose, methylcellulose, hydroxypropyl methylcellulose), hyaluronic acid, cellulose acetate phthalate, polyethylene glycol, polyvinyl alcohols or poloxamers can be added. Wetting agents or surfactants such as benzalkonium chloride, polysorbate 20, polysorbate 80, dioctyl sodium sulphosuccinate can be included. Some amino acids, alone or in combination with sodium hyaluronate may be helpful in promoting tissue reconstitution, if needed. Suitable amino acids are glycine, leucine, lysine and proline (cf. EP 1940381 B1).

Thus, the present disclosure refers also to a pharmaceutical composition for use as described before, wherein the pharmaceutical composition is a formulation for a throat spray, nose spray or eye drops.

In a further aspect of the invention a method of treatment of a coronaviral infection is disclosed, in which an effective dose of a deubiquitinase inhibitor according to the disclosure is administered to a patient in need thereof.

Tautomerism relates to a rapid intraconversion of organic compounds in which a hydrogen atom or proton formally migrates inside the compound. This is accompanied by a switch of a single bond and adjacent double bond. The single forms are called tautomers. Thus, the present patent application refers also to the use of all tautomers of the at least one deubiquitinase inhibitor according to the disclosure.

Isomer is a generic term for molecules with the same chemical formula but a different chemical structure. They can be differentiated into constitutional (structural) isomers (wherein an exchange of atoms or of a functional group occurs) and stereoisomers. Stereoisomers can be subdivided into enantiomers (non-superimposable mirror images of the same molecule) and diastereomers (the same molecule with a different configuration at one or more stereocenters). Diastereomers can be subdivided into cis/trans isomers (referring to the relative orientation of functional groups within a molecule) and on the other hand conformers (rotation about formally single bonds) and rotamers (different rotational positioning about a single bond). Thus, the present patent application refers also to the use of all isomers of the at least one deubiquitinase inhibitor according to the disclosure.

For some applications it may be desirable that isotopically enriched forms of the compounds of the invention are used, e.g. for diagnostic purposes. Thus, the present patent application refers also to such isotopically enriched forms of the compounds of the invention.

From a pharmacokinetic point of view or for a production rationale it may be preferable to use a prodrug as a dosage form. A prodrug is administered in a pharmacologically inactive form and is metabolically converted into the active form inside the body. This conversion may occur systemically or locally. Thus, the present patent application refers also to prodrugs of the compounds of the invention.

As used throughout the present application the terms “the at least one deubiquitinase inhibitor according to the disclosure” shall encompass all the aforementioned molecular variants, unless otherwise stated.

### EXAMPLES

### Example 1: PR-619 Inhibits the Replication of SARS-CoV-2 in Infected Vero-B4 Cells

In order to investigate whether PR-619 has an effect on the spread of viral infection, Western Blot (WB) analyses were carried out. Vero-B4 cells (Meyer et al. (2015) Emerg Infect Dis 21: 181-182) were infected with SARS-CoV-2 for two hours. Cells were then washed with PBS (phosphate buffer saline), provided with fresh medium containing PR-619 in a non-cytotoxic concentration (10 μM). The treatment with PR-619 was carried out over the entire experimental procedure. 3 days post infection (dpi) cells and the virus-containing supernatants were harvested. Then, a separation into a cell and a virus fraction was conducted by means of centrifugation. Virions were purified from the cell culture supernatants via a 20% sucrose cushion. Cells were washed with PBS and lysed with RIPA buffer. Protein concentrations were determined by means of Bradford protein assays and assimilated for the respective lysates. The cytosolic fraction of cell lysates was denaturized in SDS (sodium dodecyl sulfate) sample buffer, separated by SDS gel electrophoresis and transferred to a nitrocellulose membrane. SARS-CoV-2 were visualized using a convalescent serum and a horseradish peroxidase-coupled secondary reagent by means of an electrochemiluminescence reaction. Herein, an inhibition of SARS-Cov-2 replication was shown in Vero-B4 cells. At a concentration of 10 μM PR-619 showed a clear reduction of SARS-CoV-2 proteins, both in the virus (FIG. 1A) and in the cell (FIG. 1B) fraction. 10 μM chloroquine (CQN) and 10 μM hydroxychloroquine (H-CQN) (compounds known to reduce SARS-CoV-2 load) were used as positive control.

Densitometric evaluations of SARS-CoV-2 nucleoprotein in the virus fraction and spike protein S1 in the cell fraction were carried out with the analysis program AIDA®. Densitometric evaluation allows for the quantification of signal intensities in Western Blot and thus for conclusions on the quantity of a certain protein in the sample. The evaluation showed clearly that after the addition of PR-619 the generation of SARS-CoV-2 proteins is significantly inhibited, even to a higher percentage as for the same concentration of chloroquine or hydroxychloroquine (FIG. 2; FIG. 2A virus fraction, FIG. 2B cell fraction, results of 3 independent experiments).

### Example 2: The Inhibition of SARS-CoV-2 Replication in Infected Vero-B4 Cells is Corroborated by qRT-PCR

To further confirm the activity of PR-619 against SARS-CoV-2 qRT-PCR (quantitative real-time polymerase chain reaction) experiments were conducted. Therefore, virus-containing samples were quantified by real-time PCR AgPath-ID One-Step RT-PCR Kit from Ambion (Cat: 4387424) allowing reverse transcription, cDNA synthesis and PCR amplification in a single step. Samples were analyzed by 7500 software v2.3 (applied Bioscience). PCR primers were used according to 44: RdRp_fwd: 5′-GTG-ARA-TGG-TCA-TGT-GTG-GCG-G-3′ and RdRp_rev 5′-CAR-ATG-TTA-AAS-ACA-CTA-TTA-GCA-TA-C-3′. Probe was 5′-CAG-GTG-GAA-/ZEN/CCT-CAT-CAG-GAG-ATG-C-3′ (Label: FAM/IBFQ Iowa Black FQ). As positive control a specific target for E and RdRp gen of SARS-CoV2 was used and made by Integrated DNA Technologies. Control: 5′-TAA-TAC-GAC-TCA-CTA-TAG-GGT-ATT-GAG-TGA-AAT-GGT-CAT-GTG-TGG-CGG-TTC-ACT-ATA-TGT-TAA-ACC-AGG-TGG-AAC-CTC-ATC-AGG-AGA-TGC-CAC-AAC-TGC-TTA-TGC-TAA-TAG-TGT-TTT-TAA-CAT-TTG-GAA-GAG- ACA-GGT-ACG-TTA-ATA-GTT-AAT-AGC-GTA-CTT-CTT-TTT-CTT-GCT-TTC-GTG-GTA-TTC-TTG-CTA-GTT-ACA-CTA-GCC-ATC-CTT-ACT-GCG-CTT-CGA-TTG-TGT-GCG-TAC-TGC-TGC-AAT-ATT-GTT-3′. The annealing temperature was 60° C. The experimental procedure containing the infection, treatment and isolation of virions was the same as described for the Western blot analysis. The evaluation of qRT-PCR analysis clearly confirmed the results of the Western blot experiments. After the addition of PR-619 the amount of viral RNA is significantly reduced, even to a higher percentage as for the same concentration of chloroquine or hydroxychloroquine (FIG. 3, results of 3 independent experiments).

### Example 3: In Effective Concentrations PR-619 is not Cytotoxic in Vero-B4 Cell Cultures

For addressing the question whether PR-619 shows a cytotoxic effect in the abovementioned systems non-infected Vero-B4 cells were treated in parallel to the Western blot studies with increasing concentrations of PR-619 (2.5 μM, 5 μM, 10 μM, 20 μM). Toxicity was assessed with a WST assay. Herein viable cells with an intact mitochondrial succinate-tetrazolium dehydrogenase system effect an enzymatic conversion of the feebly red tetrazolium salt WST-1 (4-[3-(4-iodophenyl)-2-(4-nitrophenyl)-2H-5-tetrazolio]-1,3 benzene disulfonate) into dark red formazan. This color change can be measured photometrically in a spectrophotometer. Thus, the WST assay is a very sensitive method for measuring the toxicity of substances on the cell metabolism.

In FIG. 4A (results of 3 independent experiments) the percentage of viable cells is depicted in comparison to untreated cells. The value for untreated cells was set to 100%. For comparative reasons, the same experiment was carried out with chloroquine (FIG. 4B, results of 3 independent experiments) and hydroxychloroquine (FIG. 4C, results of 3 independent experiments) (2.5 μM, 5 μM, 10 μM, 20 μM, respectively). 1 μM staurosporine (an indolocarbazole compound from Streptomyces staurosporeus, an apoptosis inducer) was used as positive control.

It could be shown that PR-619 did not display any significant toxic effect in antivirally effective concentrations in Vero-B4 cells during an observation period of 3 days. Only at concentrations over 20 μM a clear toxic effect started.

Thus, it can be stated that the antiviral effect of PR-619 is not due to unspecific cytotoxic effects.

### Example 4: PR-619 Directly Inhibits the Papain-Like Protease (PLpro) of SARS-CoV-2

It is well known that PLpro can act as a viral deubiquitinating enzyme. It was reported that the inhibition of this virally encoded deubiquitinase interferes with the replication of the severe acute respiratory syndrome (SARS)-Coronavirus. Additionally, data showed that SARS-CoV-2 PLpro does not only play a role in processing of viral proteins and virus proliferation but has major effects on innate immunity as well. As PLpro of SARS-CoV shares a high homology with the protease of SARS-CoV-2 it can be assumed that both proteases have deubiquitinating function and therefore can be targeted by inhibitors of deubiquitinating enzymes. Thus, it was intriguing to hypothesize that PR-619 potentially interacts with SARS CoV-2 directly by blocking the activity of PLpro. To evaluate this hypothesis, recombinant SARS-CoV-2 PLpro and a specific AMC-conjugated substrate, the Ub-like protein interferon-stimulated gene 15 (ISG-15) were used. Following type I interferon stimulation, ISG-15 getting activated and was shown to regulate the immune response by IFN-γ and cytokine production and, thereby, mediate protection against a variety of viruses, amongst them Influenza A and B, Hepatitis B and C, HIV-1 and HPV-16. However, viruses have evolved countermeasures to antagonize ISG-15 and thus escape the innate immune response. SARS-CoV PLpro antagonistic activity against ISG-15 blocks the production of various cytokines involved in the activation of the innate immune response against viral infection, e.g. Type I interferon-β (IFNb) and chemokines as CXCL10 and CCL5.

To test the hypothesis that PR-619 directly blocks the activity of PLpro from SARS-CoV-2, recombinant PLpro (20 nM; R&D biosystems #E-611-050) was mixed with ISG15-Amido-4-methylcoumarin (AMC; R&D Biosystems #UL-553-050) substrate (400 nmol) in a 96-well plate with black bottom. Shortly after, protease activity was determined for 120 min using a Victor-Reader (excitation: 380 nm; emission: 460 nm). In case of treatment with PR-619, PLpro was pretreated for 30 minutes. The results clearly show that PR-619 elicits a significant and dose-dependent inhibitory effect on SARS-CoV-2 PLpro (FIG. 5A).

To analyze the statistical relevance of this observation, the area-under-the-curve (AUC), representing the SARS-CoV-2 PLpro activity in each of the three independently conducted experiments, was calculated for each concentration of PR-619 (FIG. 5B). Thereby, a dose-dependent reduction of the SARS-CoV-2 PLpro activity was measured following addition of increasing amounts of PR-619.

### Example 5: P5091, a Specific Inhibitor of USP7 and USP47, Blocks the Replication of SARS-CoV-2

We analyzed the influence of the specific USP7/USP47-inhibitor P5091 on the replication of SARS-CoV-2. Therefore, Western Blot (WB) analyses were carried out according to the experimental procedures described in Example 1.

The results of these analysis clearly showed a dose-dependent decrease in the release of viral proteins following treatment with P5091 (FIG. 6A). Densitometric analysis of 3 independent experiments revealed a significant reduction in the release of SARS-CoV-2 proteins by 90% using 1.25 μM P5091 and a complete block using 5 μM P5091 (FIG. 6B).

### Example 6: In Effective Concentrations P5091 is not Cytotoxic in Vero-B4 Cell Cultures

Cytotoxicity experiments were carried out according to Example 4. In the dose range of Example 5 P5091 showed no cytotoxic effects (FIG. 7). These results clearly demonstrate that USP7, respectively USP47 are important cellular factors in the replication cycle of SARS-CoV-2.

### Example 7: P5091 Inhibits the Activity of SARS-CoV-2 PLpro

As USP7 exhibits structural similarity with the PLpro from SARS and the PLpro from SARS-CoV-2 has an over 90% homology to the PLpro from SARS, we hypothesized that P5091 also inhibits the activity of the PLpro from SARS-CoV-2.

To check this hypothesis experiments were conducted according to the protocol of Example 4. FIG. 8A shows a dose dependent reduction of the activity of the SARS-CoV-2 PLpro following treatment with P5091. Calculation of the AUC clearly showed that the activity of the PLpro from SARS-Cov-2 was reduced by 80% following treatment with 6 μM P5091 (FIG. 8B). Thus, it can be concluded that P5091 has, in addition to the inhibition of the cellular USP7, also direct anti-viral activity by inhibiting the SARS-CoV-2 Plpro.

### FIGURES

In all Figures, * stands for p<0.05, ** for p<0.01, ***for p<0.001 and **** for p<0.0001.

FIG. 1: Western Blot bands after 3d treatment with 10 μM PR-619, 10 μM chloroquine or 10 μM hydroxychloroquine, respectively, vs. untreated cells
A: virus fraction (nucleoprotein)
B: cell fraction (spike protein S1)

FIG. 2: Densitometric evaluation of viral protein detected in Western Blot bands after 3d treatment with 10 μM PR-619, 10 μM chloroquine or 10 μM hydroxychloroquine (n=3, respectively). Untreated cells were taken as 100%.
A: virus fraction (nucleoprotein)
B: cell fraction (spike protein S1)

FIG. 3: Evaluation of qRT-PCR analysis after 3d treatment with 10 μM PR-619, 10 μM chloroquine or 10 μM hydroxychloroquine

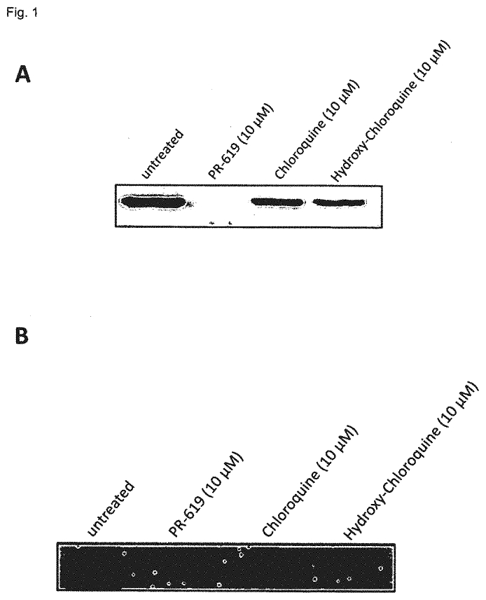

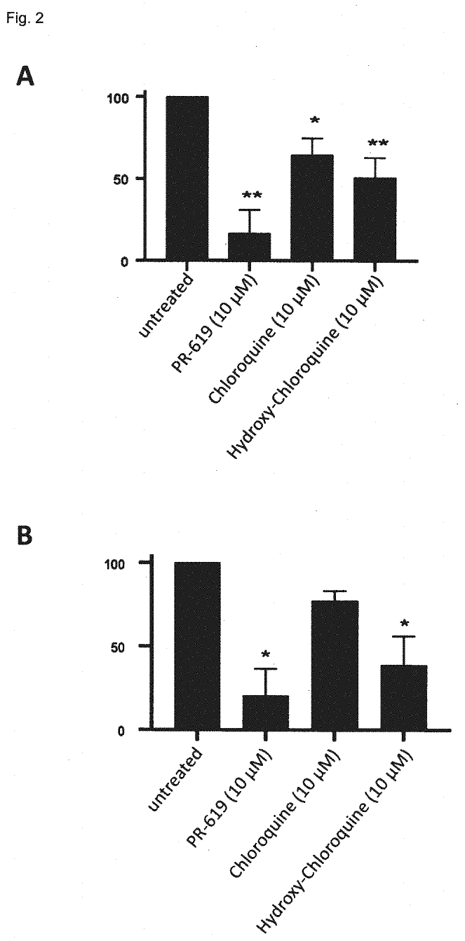

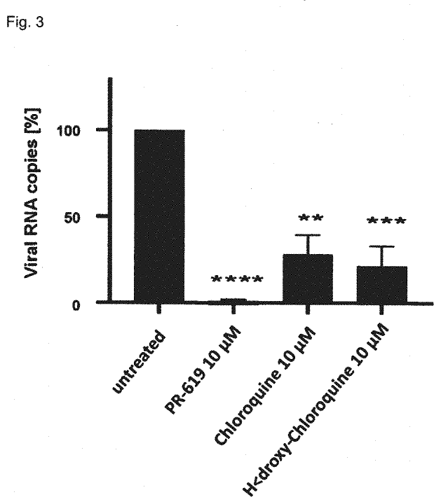

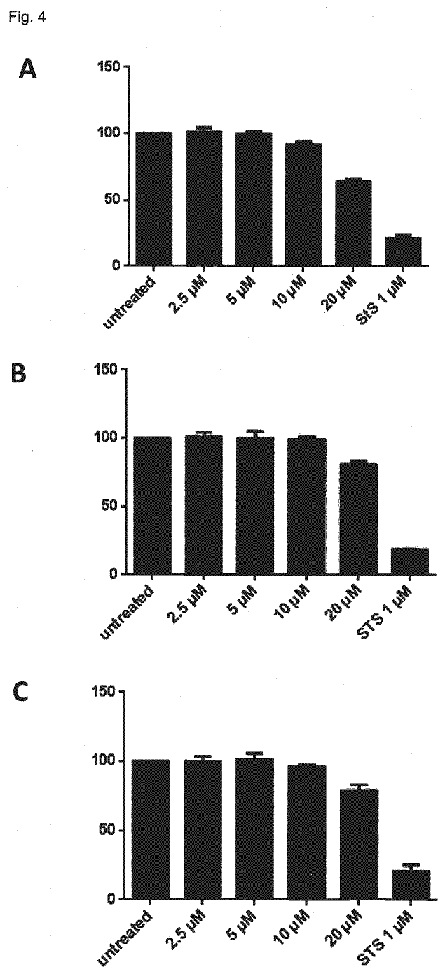

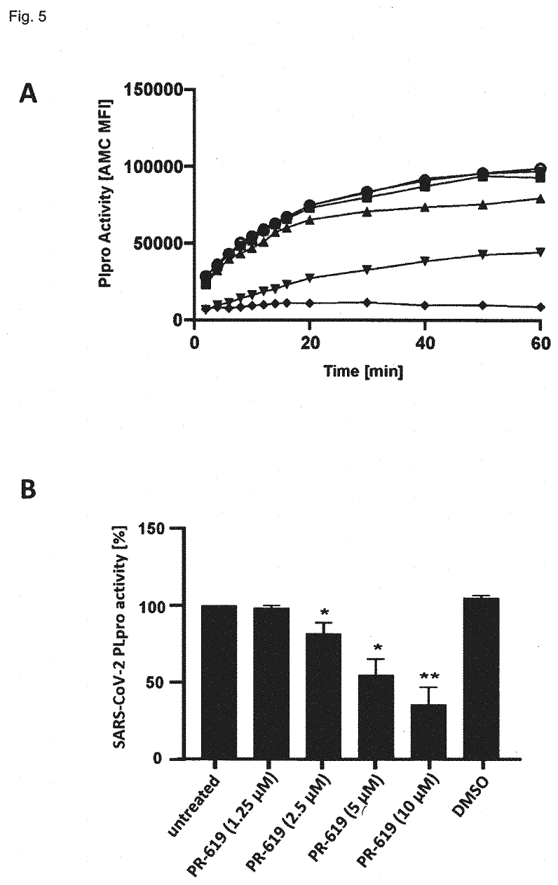

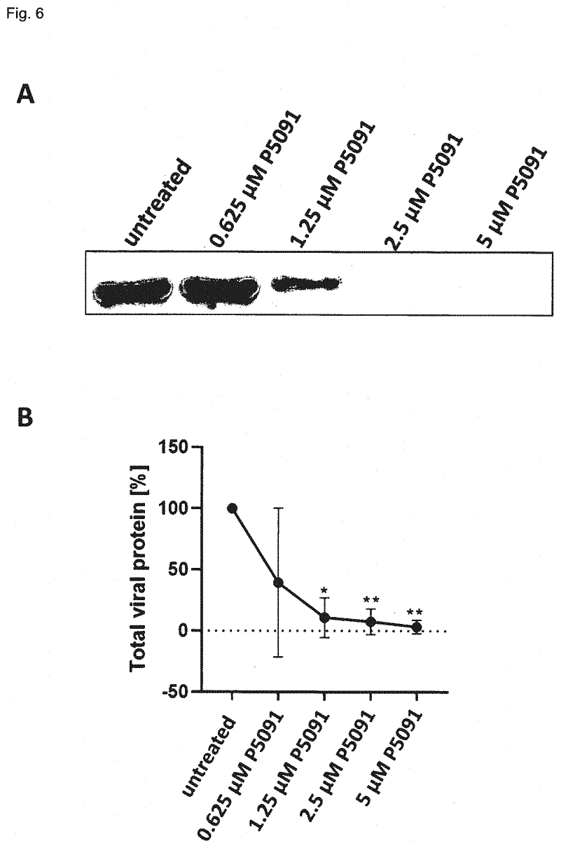

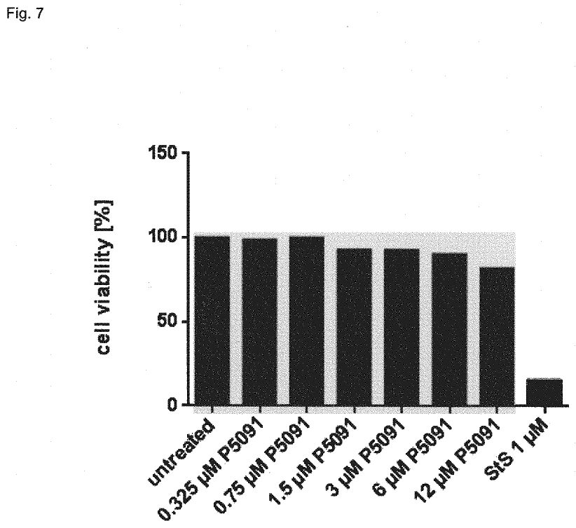

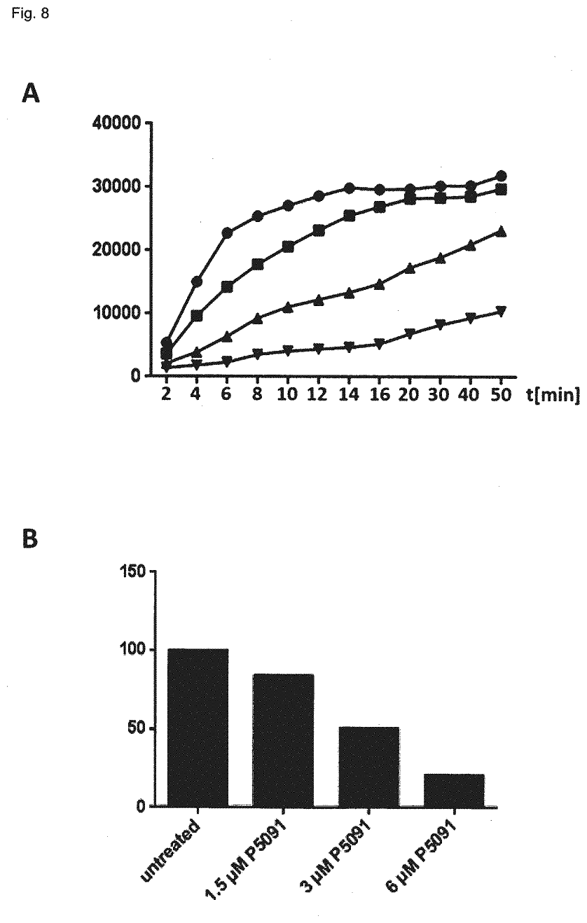

FIG. 4: Cell viability in the WST assay after PR-619 treatment. Untreated cells were taken as 100%. Staurosporine (StS) was used as positive control. (n=3, respectively)
A: PR-619
B: chloroquine
C: hydroxychloroquine

FIG. 5: Evaluation of the effects of PR-619 on SARS-CoV-2 PLpro (n=3, respectively)
A: inhibitory effect on SARS-CoV-2 PLpro. DMSO was used as control.
B: SARS-CoV-2 PLpro activity calculated for each concentration
   [symbol] untreated
   [symbol] 1.25 μM PR-619
   [symbol] 2.5 μM PR-619
   [symbol] 5 μM PR-619
   [symbol] 10 μM PR-619
   [symbol] DMSO

FIG. 6: Evaluation of the effects of different concentrations of P5091
A: Western Blot bands after 3d treatment with P5091 (total viral protein)
B: Densitometric evaluation of viral protein detected in Western Blot bands after 3d treatment with P5091. Untreated cells were taken as 100%.

FIG. 7: Cell viability in the WST assay after P5091 treatment. Untreated cells were taken as 100%. Staurosporine (StS) was used as positive control (n=1)

FIG. 8: Evaluation of the effects of P5091 on SARS-CoV-2 PLpro (n=3, respectively)
A: inhibitory effect on SARS-CoV-2 PLpro
B: SARS-CoV-2 PLpro activity calculated for each concentration
   [symbol] 6 μM P5091
   [symbol] 3 μM P5091
   [symbol] 1.5 μM P5019
   [symbol] untreated

1. A method of treating an individual having a SARS-CoV-2 infection by administering to such individual a pharmaceutically effective amount of an inhibitor of human deubiquitinases USP7 and/or USP47 or one of its pharmaceutically acceptable salts, hydrates or solvates.

2. The method of claim 1, wherein said inhibitor is a pyridine-3,5-(bis)thiocyanate according to general formula I 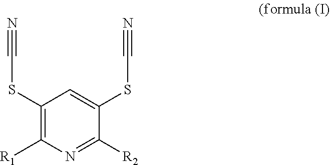
wherein
R₁ and R₂ each independently from one another is —H, —OH, —NHR₃, —NR₃R₄, a substituted or non-substituted linear or ramified alkyl residue with 1 to 3 carbon atoms, —CO—OCH₃, —CO—OC₂H₅, —CO—NH₂, —NH₂, —NO₂, —Cl, —Br, —F, or —SO₂H;
R₃ and R₄ each independently from one another is —OH, —CH₃, —C₂H₅, —CH₂OH, —CHO, —COOH, —CO—CH₃, or —CO—NH₂,
or one of its pharmaceutically acceptable salts, hydrates and solvates.

3. The method of claim 2, wherein said pyridine-3,5-(bis)thiocyanate is 2,6-diaminopyridine-3,5-bis(thiocyanate).

4. The method of claim 1, wherein said inhibitor is a disubstituted 4-nitro-thiophene according to general formula III 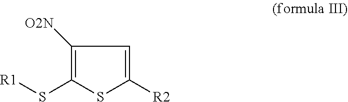
wherein
R1 is phenyl, 2-chlorophenyl, 3-chlorophenyl, 4-chlorophenyl, 2,3-dichlorophenyl, 3,5-dichlorophenyl, 2,4-dichlorophenyl or 2,4-difluorophenyl, and
R2 is acetyl, 1-hydroxyethyl, ethyl or n-butyl,
or one of its pharmaceutically acceptable salts, hydrates and solvates.

5. The method of claim 4, wherein said disubstituted 4-nitro-thiophene is 1-[5-[(2,3-dichlorophenyl)thio]-4-nitro-2-thienyl]-ethanone.

6. The method of claim 1, wherein said inhibitor is selected from a group consisting of P22077, ADC-01, ADC-03, HBX41108, HBX19818, HBX 28258, NSC 632839, NSC 144303, GNE-6640, GNE-6776, FT671, FT827, XL188, XL177a, XL024, XL058, XL041, 4-cyano-5-[(3,5-dichloro-4-pyridinyl)thio]-N-[4-(methylsulfonyl)phenyl]-2-thiophenecarboxamide and parthenolide.

7. (canceled)

8. A method of treating an individual having a SARS-CoV-2 infection by administering to such individual a pharmaceutically effective amount of a pharmaceutical composition comprising one of the inhibitors of claim 1, a carrier and at least one pharmaceutically acceptable excipient.

9. The method of claim 8, wherein the pharmaceutical composition is a formulation for oral administration.

10. The method of claim 8, wherein the pharmaceutical composition is a formulation for inhalatory administration.

11. The method of claim 10, wherein the inhalatory administration is carried out by means of a vibrating mesh nebulizer.

12. The method of claim 8, wherein the pharmaceutical composition is a liquid dosage form.

13. The method of claim 8, wherein the pharmaceutical composition is a formulation for sublingual tablets.

14. The method of claim 8, wherein the pharmaceutical composition is a formulation for a throat spray, nose spray or eye drops.

15. (canceled)
